SEQUENCE LISTING (16 sequences)

SEQ ID NO: 1 - DNA, 1519 bp, Megasphaera massiliensis
  Megasphaera massiliensis gene for 16S ribosomal RNA, partial sequence, strain NP3 - JX424772.1
agagtttgatcctggctcaggacgaacgctggcggcgtgcttaacacatgcaagtcgaacgagaagagatgagaagcttgcttcttatcaattcgagtggcaaacgggtgagtaacgcgtaagcaacctgcccttcagatggggacaacagctggaaacggctgctaataccgaatacgttctttccgccgcatgacgggaagaagaaagggaggccttcgggctttcgctggaggaggggcttgcgtctgattagctagttggaggggtaacggcccaccaaggcgacgatcagtagccggtctgagaggatgaacggccacattgggactgagacacggcccagactcctacgggaggcagcagtggggaatcttccgcaatggacgaaagtctgacggagcaacgccgcgtgaacgatgacggccttcgggttgtaaagttctgttatatgggacgaacagggcatcggttaatacccggtgtctttgacggtaccgtaagagaaagccacggctaactacgtgccagcagccgcggtaatacgtaggtggcaagcgttgtccggaattattgggcgtaaagggcgcgcaggcggcatcgcaagtcggtcttaaaagtgcggggcttaaccccgtgaggggaccgaaactgtgaagctcgagtgtcggagaggaaagcggaattcctagtgtagcggtgaaatgcgtagatattaggaggaacaccagtggcgaaagcggctttctggacgacaactgacgctgaggcgcgaaagccaggggagcaaacgggattagataccccggtagtcctggccgtaaacgatggatactaggtgtaggaggtatcgactccttctgtgccggagttaacgcaataagtatcccgcctggggagtacggccgcaaggctgaaactcaaaggaattgacgggggcccgcacaagcggtggagtatgtggtttaattcgacgcaacgcgaagaaccttaccaagccttgacattgattgctacggaaagagatttccggttcttcttcggaagacaagaaaacaggtggtgcacggctgtcgtcagctcgtgtcgtgagatgttgggttaagtcccgcaacgagcgcaacccctatcttctgttgccagcacctcgggtggggactcagaagagactgccgcagacaatgcggaggaaggcggggatgacgtcaagtcatcatgccccttatggcttgggctacacacgtactacaatggctcttaatagagggacgcgaaggagcgatccggagcaaaccccaaaaacagagtcccagttcggattgcaggctgcaactcgcctgcatgaagcaggaatcgctagtaatcgcaggtcagcatactgcggtgaatacgttcccgggccttgtacacaccgcccgtcacaccacgaaagtcattcacacccgaagccggtgaggcaaccgcaaggaaccagccgtcgaaggtgggggcgatgattggggtgaagtcgtaacaaggt

SEQ ID NO: 2 - DNA, 1398 bp, Megasphaera massiliensis
  consensus 16S rRNA gene sequence for Megasphaera massiliensis strain MRx0029
tgagaagcttgcttcttatcgattctagtggcaaacgggtgagtaacgcgtaagcaacctgcccttcagatggggacaacagctggaaacggctgctaataccgaatacgttctttccgccgcatgacgggaagaagaaagggaggccttcgggctttcgctggaggaggggcttgcgtctgattagctagttggaggggtaacggcccaccaaggcgacgatcagtagccggtctgagaggatgaacggccacattgggactgagacacggcccagactcctacgggaggcagcagtggggaatcttccgcaatggacgaaagtctgacggagcaacgccgcgtgaacgatgacggccttcgggttgtaaagttctgttatatgggacgaacaggacatcggttaatacccggtgtctttgacggtaccgtaagagaaagccacggctaactacgtgccagcagccgcggtaatacgtaggtggcaagcgttgtccggaattattgggcgtaaagggcgcgcaggcggcatcgcaagtcggtcttaaaagtgcggggcttaaccccgtgaggggaccgaaactgtgaagctcgagtgtcggagaggaaagcggaattcctagtgtagcggtgaaatgcgtagatattaggaggaacaccagtggcgaaagcggctttctggacgacaactgacgctgaggcgcgaaagccaggggagcaaacgggattagataccccggtagtcctggccgtaaacgatggatactaggtgtaggaggtatcgactccttctgtgccggagttaacgcaataagtatcccgcctggggagtacggccgcaaggctgaaactcaaaggaattgacgggggcccgcacaagcggtggagtatgtggtttaattcgacgcaacgcgaagaaccttaccaagccttgacattgattgctacggaaagagatttccggttcttcttcggaagacaagaaaacaggtggtgcacggctgtcgtcagctcgtgtcgtgagatgttgggttaagtcccgcaacgagcgcaacccctatcttctgttgccagcacctcgggtggggactcagaagagactgccgcagacaatgcggaggaaggcggggatgacgtcaagtcatcatgccccttatggcttgggctacacacgtactacaatggctcttaatagagggaagcgaaggagcgatccggagcaaaccccaaaaacagagtcccagttcggattgcaggctgcaactcgcctgcatgaagcaggaatcgctagtaatcgcaggtcagcatactgcggtgaatacgttcccgggccttgtacacaccgcccgtcacaccacgaaagtcattcacacccgaagccggtgaggcaaccgcaag

SEQ ID NO: 3 - DNA, 20 bp, Artificial sequence
  Description of Artificial Sequence TPH1 forward primer
aaagagcgtacaggtttttc

SEQ ID NO: 4 - DNA, 20 bp, Artificial sequence
  Description of Artificial Sequence TPH1 reverse primer
gtctcacatattgagtgcag

SEQ ID NO: 5 - DNA, 20 bp, Artificial sequence
  Description of Artificial Sequence Beta-actin forward primer
gatcaagatcattgctcctc

SEQ ID NO: 6 - DNA, 20 bp, Artificial sequence
  Description of Artificial Sequence Beta-actin reverse primer
ttgtcaagaaagggtgtaac

SEQ ID NO: 7 - DNA, 19 bp, Artificial sequence
  Description of Artificial Sequence SLC6A4 forward primer
aatctgccgattttcaaag

SEQ ID NO: 8 - DNA, 21 bp, Artificial sequence
  Description of Artificial Sequence SLC6A4 reverse primer
gtgttgtagtaggaagcaatg

SEQ ID NO: 9 - DNA, 20 bp, Artificial sequence
  Description of Artificial Sequence TPH2 forward primer
cactattgtgacgctgaatc

SEQ ID NO: 10 - DNA, 20 bp, Artificial sequence
  Description of Artificial Sequence TPH2 reverse primer
agctcagaaccatacatgag

SEQ ID NO: 11 - DNA, 1409 bp, Megasphaera
  consensus 16S rRNA sequence for the Megasphaera strain deposited under accession number NCIMB 43385
ggctggttccttgcggttgcctcaccggcttcgggtgtgaatgactttcgtggtgtgacgggcggtgtgtacaaggcccgggaacgtattcaccgcagtatgctgacctgcgattactagcgattcctgcttcatgcaggcgagttgcagcctgcaatccgaactgggactctgtttttggggtttgctccggatcgctccttcgcttccctctattaagagccattgtagtacgtgtgtagcccaagccataaggggcatgatgacttgacgtcatccccgccttcctccgcattgtctgcggcagtctcttctgagtccccacccttagtgctggcaacagaagataggggttgcgctcgttgcgggacttaacccaacatctcacgacacgagctgacgacagccgtgcaccacctgttttcttgtcttccgaagaagaaccggaaatctctttccgtagcaatcaatgtcaaggcttggtaaggttcttcgcgttgcgtcgaattaaaccacatactccaccgcttgtgcgggcccccgtcaattcctttgagtttcagccttgcggccgtactccccaggcgggatacttattgcgttaactccggcacagaaggagtcgatacctcctacacctagtatccatcgtttacggccaggactaccggggtatctaatcccgtttgctcccctggctttcgcgcctcagcgtcagttgtcgtccagaaagccgctttcgccactggtgttcctcctaatatctacgcatttcaccgctacactaggaattccgctttcctctccgacactcgagcttcacagtttcggtcccctcacggggttaagccccgcacttttaagaccgacttgcgatgccgcctgcgcgccctttacgcccaataattccggacaacgcttgccacctacgtattaccgcggctgctggcacgtagttagccgtggctttctcttacggtaccgtcagggataacgggtattgaccgctatcctgttcgtcccatataacagaactttacaacccgaaggccgtcatcgttcacgcggcgttgctccgtcagactttcgtccattgcggaagattccccactgctgcctcccgtaggagtctgggccgtgtctcagtcccaatgtggccgttcatcctctcagaccggctactgatcgtcgccttggtgggccgttacccctccaactagctaatcagacgcaagcccctcctccagcgaaagcccgaaggcctccctttcttcatcccgtcatgcggcggaaagaacgtattcggtattagcagccgtttccagctgttgtccccatctgaagggcaggttgcttacgcgttactcacccgtttgccactcgaattgataagaagcaagcttctcatc

SEQ ID NO: 12 - DNA, 1415 bp, megasphaera massiliensis
  consensus 16s rRNA sequence for the megasphaera massiliensis strain deposited under accession number ncimb 43388
ggctggttccttgcggttgcctcaccggcttcgggtgtgaatgactttcgtggtgtgacgggcggtgtgtacaaggcccgggaacgtattcaccgcagtatgctgacctgcgattactagcgattcctgcttcatgcaggcgagttgcagcctgcaatccgaactgggactctgtttttggggtttgctccggatcgctccttcgcttccctctattaagagccattgtagtacgtgtgtagcccaagccataaggggcatgatgacttgacgtcatccccgccttcctccgcattgtctgcggcagtctcttctgagtccccacccgaggtgctggcaacagaagataggggttgcgctcgttgcgggacttaacccaacatctcacgacacgagctgacgacagccgtgcaccacctgttttcttgtcttccgaagaagaaccggaaatctctttccgtagcaatcaatgtcaaggcttggtaaggttcttcgcgttgcgtcgaattaaaccacatactccaccgcttgtgcgggcccccgtcaattcctttgagtttcagccttgcggccgtactccccaggcgggatacttattgcgttaactccggcacagaaggagtcgatacctcctacacctagtatccatcgtttacggccaggactaccggggtatctaatcccgtttgctcccctggctttcgcgcctcagcgtcagttgtcgtccagaaagccgctttcgccactggtgttcctcctaatatctacgcatttcaccgctacactaggaattccgctttcctctccgacactcgagcttcacagtttcggtcccctcacggggttaagccccgcacttttaagaccgacttgcgatgccgcctgcgcgccctttacgcccaataattccggacaacgcttgccacctacgtattaccgcggctgctggcacgtagttagccgtggctttctcttacggtaccgtcaaagacaccgggtattaaccgatgtcctgttcgtcccatataacagaactttacaacccgaaggccgtcatcgttcacgcggcgttgctccgtcagactttcgtccattgcggaagattccccactgctgcctcccgtaggagtctgggccgtgtctcagtcccaatgtggccgttcatcctctcagaccggctactgatcgtcgccttggtgggccgttacccctccaactagctaatcagacgcaagcccctcctccagcgaaagcccgaaggcctccctttcttcttcccgtcatgcggcggaaagaacgtattcggtattagcagccgtttccagctgttgtccccatctgaagggcaggttgcttacgcgttactcacccgtttgccactagaatcgataagaagcaagcttctcatgtcttct

SEQ ID NO: 13 - DNA, 1433 bp, Megasphaera massilliensis
  consensus 16S rRNA sequence for the Megasphaera massilliensis strain deposited under accession number NCIMB 43389
cgacggctggttccttgcggttgcctcaccggcttcgggtgtgaatgactttcgtggtgtgacgggcggtgtgtacaaggcccgggaacgtattcaccgcagtatgctgacctgcgattactagcgattcctgcttcatgcaggcgagttgcagcctgcaatccgaactgggactctgtttttggggtttgctccggatcgctccttcgcttccctctattaagagccattgtagtacgtgtgtagcccaagccataaggggcatgatgacttgacgtcatccccgccttcctccgcattgtctgcggcagtctcttctgagtccccacccgaggtgctggcaacagaagataggggttgcgctcgttgcgggacttaacccaacatctcacgacacgagctgacgacagccgtgcaccacctgttttcttgtcttccgaagaagaaccggaaatctctttccgtagcaatcaatgtcaaggcttggtaaggttcttcgcgttgcgtcgaattaaaccacatactccaccgcttgtgcgggcccccgtcaattcctttgagtttcagccttgcggccgtactccccaggcgggatacttattgcgttaactccggcacagaaggagtcgatacctcctacacctagtatccatcgtttacggccaggactaccggggtatctaatcccgtttgctcccctggctttcgcgcctcagcgtcagttgtcgtccagaaagccgctttcgccactggtgttcctcctaatatctacgcatttcaccgctacactaggaattccgctttcctctccgacactcgagcttcacagtttcggtcccctcacggggttaagccccgcacttttaagaccgacttgcgatgccgcctgcgcgccctttacgcccaataattccggacaacgcttgccacctacgtattaccgcggctgctggcacgtagttagccgtggctttctcttacggtaccgtcaaagacaccgggtattaaccgatgccctgttcgtcccatataacagaactttacaacccgaaggccgtcatcgttcacgcggcgttgctccgtcagactttcgtccattgcggaagattccccactgctgcctcccgtaggagtctgggccgtgtctcagtcccaatgtggccgttcatcctctcagaccggctactgatcgtcgccttggtgggccgttacccctccaaccagctaatcagacgcaagcccctcctccagcgaaagcccgaaggcctccctttcttcttcccgtcatgcggcggaaagaacgtattcggtattagcagccgtttccagctgttgtccccatctgaagggcaggttgcttacgcgttactcacccgtttgccactagaatcgataagaagcaagcttctcatgtcttctcgttcgacttgcat

SEQ ID NO: 14 - DNA, 1431 bp, Megasphaera
  consensus 16S rRNA sequence for the Megasphaera strain deposited under accession number NCIMB 43386
cgacggctggttccttgcggttgcctcaccggcttcgggtgtgaatgactttcgtggtgtgacgggcggtgtgtacaaggcccgggaacgtattcaccgcagtatgctgacctgcgattactagcgattcctgcttcatgcaggcgagttgcagcctgcaatccgaactgggactctgtttttggggtttgctccggatcgctccttcgcttccctctattaagagccattgtagtacgtgtgtagcccaagccataaggggcatgatgacttgacgtcatccccgccttcctccgcattgtctgcggcagtctcttctgagtccccacccttagtgctggcaacagaagataggggttgcgctcgttgcgggacttaacccaacatctcacgacacgagctgacgacagccgtgcaccacctgttttcttgtcttccgaagaagaaccggaaatctctttccgtagcaatcaatgtcaaggcttggtaaggttcttcgcgttgcgtcgaattaaaccacatactccaccgcttgtgcgggcccccgtcaattcctttgagtttcagccttgcggccgtactccccaggcgggatacttattgcgttaactccggcacagaaggagtcgatacctcctacacctagtatccatcgtttacggccaggactaccggggtatctaatcccgtttgctcccctggctttcgcgcctcagcgtcagttgtcgtccagaaagccgctttcgccactggtgttcctcctaatatctacgcatttcaccgctacactaggaattccgctttcctctccgacactcgagcttcacagtttcggtcccctcacggggttaagccccgcacttttaagaccgacttgcgatgccgcctgcgcgccctttacgcccaataattccggacaacgcttgccacctacgtattaccgcggctgctggcacgtagttagccgtggctttctcttacggtaccgtcagggataacgggtattgaccgctatcctgttcgtcccatataacagaactttacaacccgaaggccgtcatcgttcacgcggcgttgctccgtcagactttcgtccattgcggaagattccccactgctgcctcccgtaggagtctgggccgtgtctcagtcccaatgtggccgttcatcctctcagaccggctactgatcgtcgccttggtgggccgttacccctccaactagctaatcagacgcaagcccctcctccagcgaaagcccgaaggcctccctttcttcatcccgtcatgcggcggaaagaacgtattcggtattagcagccgtttccagctgttgtccccatctgaagggcaggttgcttacgcgttactcacccgtttgccactcgaattgataagaagcaagcttctcatctcttctcgttcgactgca

SEQ ID NO: 15 - DNA, 1434 bp, Megasphaera
  consensus 16S rRNA sequence for the Megasphaera strain deposited under accession number NCIMB 43387
tcgaacggctggttccttgcggttgcctcaccggcttcgggtgtgaatgactttcgtggtgtgacgggcggtgtgtacaaggcccgggaacgtattcaccgcagtatgctgacctgcgattactagcgattcctgcttcatgcaggcgagttgcagcctgcaatccgaactgggactctgtttttggggtttgctccggatcgctccttcgcttccctctattaagagccattgtagtacgtgtgtagcccaagccataaggggcatgatgacttgacgtcatccccgccttcctccgcattgtctgcggcagtctcttctgagtccccacccttagtgctggcaacagaagataggggttgcgctcgttgcgggacttaacccaacatctcacgacacgagctgacgacagccgtgcaccacctgttttcttgtcttccgaagaagaaccggaaatctctttccgtagcaatcaatgtcaaggcttggtaaggttcttcgcgttgcgtcgaattaaaccacatactccaccgcttgtgcgggcccccgtcaattcctttgagtttcagccttgcggccgtactccccaggcgggatacttattgcgttaactccggcacagaaggagtcgatacctcctacacctagtatccatcgtttacggccaggactaccggggtatctaatcccgtttgctcccctggctttcgcgcctcagcgtcagttgtcgtccagaaagccgctttcgccactggtgttcctcctaatatctacgcatttcaccgctacactaggaattccgctttcctctccgacactcgagcttcacagtttcggtcccctcacggggttaagccccgcacttttaagaccgacttgcgatgccgcctgcgcgccctttacgcccaataattccggacaacgcttgccacctacgtattaccgcggctgctggcacgtagttagccgtggctttctcttacggtaccgtcagggataacgggtattgaccgctatcctgttcgtcccatataacagaactttacaacccgaaggccgtcatcgttcacgcggcgttgctccgtcagactttcgtccattgcggaagattccccactgctgcctcccgtaggagtctgggccgtgtctcagtcccaatgtggccgttcatcctctcagaccggctactgatcgtcgccttggtgggccgttacccctccaactagctaatcagacgcaagcccctcctccagcgaaagcccgaaggcctccctttcttcatcccgtcatgcggcggaaagaacgtattcggtattagcagccgtttccagctgttgtccccatctgaagggcaggttgcttacgcgttactcacccgtttgccactcgaattgataagaagcaagcttctcatctcttctcgttcgacttgca

SEQ ID NO: 16 - DNA, 1525 bp, Megasphaera elsdenii
  consensus 16S rRNA gene sequence for Megasphaera elsdenii strain deposited under accession number NCIMB 8927
agagtttgatcctggctcaggacgaacgctggcggcgtgcttaacacatgcaagtcgaacgagaagagatgagaagcttgcttcttatcgattcgagtggcaaacgggtgagtaacgcgtaagcaacctgcccttcagatggggacaacagctggaaacggctgctaataccgaatacgttctttttgtcgcatggcagagggaagaaagggaggctcttcggagctttcgctgaaggaggggcttgcgtctgattagctagttggaggggtaacggcccaccaaggcgacgatcagtagccggtctgagaggatgaacggccacattgggactgagacacggcccagactcctacgggaggcagcagtggggaatcttccgcaatggacgaaagtctgacggagcaacgccgcgtgaacgatgacggccttcgggttgtaaagttctgttatacgggacgaatggcgtagcggtcaatacccgttacgagtgacggtaccgtaagagaaagccacggctaactacgtgccagcagccgcggtaatacgtaggtggcaagcgttgtccggaattattgggcgtaaagggcgcgcaggcggcgtcgtaagtcggtcttaaaagtgcggggcttaaccccgtgaggggaccgaaactgcgatgctagagtatcggagaggaaagcggaattcctagtgtagcggtgaaatgcgtagatattaggaggaacaccagtggcgaaagcggctttctggacgacaactgacgctgaggcgcgaaagccaggggagcaaacgggattagataccccggtagtcctggccgtaaacgatggatactaggtgtaggaggtatcgaccccttctgtgccggagttaacgcaataagtatcccgcctggggagtacggccgcaaggctgaaactcaaaggaattgacgggggcccgcacaagcggtggagtatgtggtttaattcgacgcaacgcgaagaaccttaccaagccttgacattgattgctatggatagagatatccagttcctcttcggaggacaagaaaacaggtggtgcacggctgtcgtcagctcgtgtcgtgagatgttgggttaagtcccgcaacgagcgcaacccctatcttctgttaccagcggttcggccggggactcaggagagactgccgcagacaatgcggaggaaggcggggatgacgtcaagtcatcatgccccttatggcttgggctacacacgtactacaatggctcttaatagagggaagcgaaggagcgatccggagcaaaccccaaaaacagagtcccagttcggattgcaggctgcaactcgcctgcatgaagcaggaatcgctagtaatcgcaggtcagcatactgcggtgaatacgttcccgggccttgtacacaccgcccgtcacaccacgaaagtcattcacacccgaagccggtgaggtaaccttttggagccagccgtccaaggtgggggcgatgattggggtgaagtcgtaacaaggtaacc

(19) United States
(12) Patent Application Publication
(10) Pub. No.: US 2021/0361722 A1
(43) Pub. Date: Nov. 25, 2021

(54) COMPOSITIONS COMPRISING BACTERIAL STRAINS

(71) Applicant: 4D Pharma Research Limited, Aberdeen (GB)

(72) Inventors: Imke ELISABETH MULDER, Aberdeen (GB); Anna ETTORRE, Aberdeen (GB); Suaad AHMED, Aberdeen (GB); John CRYAN, Cork (IE); Ted DINAN, Cobh (IE)

(21) Appl. No.: 17/342,579

(22) Filed: Jun. 9, 2021

Related U.S. Application Data
(63) Continuation of application No. PCT/EP2019/084991, filed on Dec. 12, 2019.

(30) Foreign Application Priority Data
Dec. 12, 2018 (EP) 18212096.4
May 13, 2019 (GB) 1906728.9
Oct. 14, 2019 (GB) 1914852.7

(51) Int. Cl. A61K 35/741; A61P 25/24; A61K 45/06; C12N 1/20

(52) U.S. Cl. CPC A61K 35/741; A61P 25/24; C12R 2001/01; C12N 1/205; A61K 45/06

(57) ABSTRACT

The invention provides a composition comprising a bacterial strain of the genus Megasphaera, for use in the treatment or prevention of a disorder characterised by serotonin deficiency.

### CROSS-REFERENCE

This application is a continuation of International Application No. PCT/EP2019/084991, filed Dec. 12, 2019, which claims the benefit of European Application No. 18212096.4, filed Dec. 12, 2018, Great Britain Application No. 1906728.9, filed May 13, 2019, and Great Britain Application No. 1914852.7, filed Oct. 14, 2019, all of which are hereby incorporated by reference in their entirety.

### SEQUENCE LISTING

The instant application contains a Sequence Listing which has been submitted electronically in ASCII format and is hereby incorporated by reference in its entirety. Said ASCII copy, created on Jun. 2, 2021, is named 56708_754_301_SL.txt and is 17,935 bytes in size.

### TECHNICAL FIELD

This invention is in the field of compositions comprising bacterial strains isolated from the mammalian digestive tract and the use of such compositions in the treatment of disease.

### BACKGROUND TO THE INVENTION

The human intestine is thought to be sterile in utero, but it is exposed to a large variety of maternal and environmental microbes immediately after birth. Thereafter, a dynamic period of microbial colonization and succession occurs, which is influenced by factors such as delivery mode, environment, diet and host genotype, all of which impact upon the composition of the gut microbiota, particularly during early life. Subsequently, the microbiota stabilizes and becomes adult-like [1]. The human gut microbiota contains more than 500-1000 different phylotypes belonging essentially to two major bacterial divisions, the Bacteroidetes and the Firmicutes [2]. The successful symbiotic relationships arising from bacterial colonization of the human gut have yielded a wide variety of metabolic, structural, protective and other beneficial functions. The enhanced metabolic activities of the colonized gut ensure that otherwise indigestible dietary components are degraded with release of by-products providing an important nutrient source for the host. Similarly, the immunological importance of the gut microbiota is well-recognized and is exemplified in germfree animals which have an impaired immune system that is functionally reconstituted following the introduction of commensal bacteria [3-5].

Dramatic changes in microbiota composition have been documented in gastrointestinal disorders such as inflammatory bowel disease (IBD). For example, the levels of Clostridium cluster XIVa bacteria are reduced in IBD patients whilst numbers of E. coli are increased, suggesting a shift in the balance of symbionts and pathobionts within the gut [6-9].

In recognition of the potential positive effect that certain bacterial strains may have on the animal gut, various strains have been proposed for use in the treatment of various diseases (see, for example, [10-13]). Also, certain strains, including mostly Lactobacillus and Bifidobacterium strains, have been proposed for use in treating various inflammatory and autoimmune diseases that are not directly linked to the intestines (see [14] and [15] for reviews). However, the relationship between different diseases and different bacterial strains, and the precise effects of particular bacterial strains on the gut and at a systemic level and on any particular types of diseases are poorly characterised.

Recently, there has been increased interest in the art regarding alterations in the gut microbiome that may play a pathophysiological role in human psychiatric disorders. Preclinical and clinical evidence are strongly suggesting a link between mental health problems and the microbiota [16]. A growing body of preclinical literature has demonstrated bidirectional signalling between the brain and the gut microbiome, involving multiple neurocrine and endocrine signalling systems. Probiotics have also been suggested to act as delivery vehicles for neurochemicals that influence extra-intestinal aspects of physiology. However, these studies have failed to identify the mechanisms by which bacteria affect neurophysiological processes and have not shown any useful effects for any bacteria in particular. This indicates that, at present, the practical effect of the link between the microbiome and human psychiatric disorders is poorly characterised. Accordingly, more direct analytical studies are required to identify the therapeutic impact of altering the microbiome on psychiatric disorders.

There is a requirement in the art for new methods of treating psychiatric disorders. There is also a requirement for the potential effects of gut bacteria to be characterised so that new therapies using gut bacteria can be developed.

### SUMMARY OF THE INVENTION

The inventors have developed new therapies for treating and preventing disorders characterised by serotonin deficiency. In particular, the inventors have developed new therapies for treating and preventing psychiatric disorders characterised by serotonin deficiency. Serotonin is a monoamine transmitter that, inter alia, contributes to feelings of well-being and happiness. A deficiency in serotonin in a subject can therefore, amongst other things, exacerbate deregulation of mood, appetite and/or sleep in the subject.

As shown in the examples, the inventors have identified that a composition comprising a bacterial strain of the genus Megasphaera upregulates the expression of tryptophan hydroxylase 1 and 2 in neurons. Tryptophan hydroxylases are enzymes involved in the production of serotonin. Thus, the composition of the invention may be useful in increasing the amount of serotonin available in the neurons of a subject, by increasing the production of serotonin via the tryptophan hydroxylase pathway, thereby treating or preventing disorders characterised by serotonin deficiency.

The inventors have also shown that a composition comprising a bacterial strain of the genus Megasphaera upregulates the expression of Serotonin Transporter (SERT) gene SLC6A4. SERT is a type of monoamine transporter protein that transports serotonin from the synaptic cleft to a presynaptic neuron. SERT is a marker of serotonergic neurons. Thus, the composition of the invention may be effective in promoting the differentiation of naïve neurons into serotonergic neurons in vivo. This may be particularly beneficial in the treatment of disorders characterised by serotonin deficiency, since serotonergic neurons produce serotonin. Therefore, increasing the number of serotonin-producing neurons in a subject may increase the production of serotonin, and therefore alleviate serotonin deficiency characterising the disorder.

The inventors have also shown that a composition comprising a bacterial strain of the genus Megasphaera increases secretion of serotonin in vivo. Thus, the inventors have found that the composition of the invention may be used to advantageously increase the bioavailability of serotonin in vivo. In particular, the inventors have found that the composition of the invention increases the bioavailability of serotonin in vivo. Therefore, the composition of the invention may be useful in the treatment or prevention of a disorder, in particular a psychiatric disorder, characterised by serotonin deficiency.

In a first embodiment, the invention provides a composition comprising a bacterial strain of the genus Megasphaera, for use in in a method of treating or preventing a disorder characterised by serotonin deficiency. In some embodiments, the composition of the invention is for use in a method of treating or preventing a psychiatric disorder characterised by serotonin deficiency.

In particular embodiments, the invention provides a composition comprising a bacterial strain of the genus Megasphaera, for use in a method of treating or preventing a disorder selected from the group consisting of: depression, anxiety, post-traumatic stress disorder and obsessive compulsive disorder.

In preferred embodiments, the invention provides a composition comprising a bacterial strain of the genus Megasphaera, for use in a method of treating or preventing depression.

In preferred embodiments of the invention, the bacterial strain in the composition is of Megasphaera massiliensis. Closely related strains may also be used, such as bacterial strains that have a 16S rRNA gene sequence that is at least 95%, 96%, 97%, 98%, 99%, 99.5% or 99.9% identical to the 16S rRNA gene sequence of a bacterial strain of Megasphaera massiliensis. Preferably, the bacterial strain has a 16S rRNA gene sequence that is at least 95%, 96%, 97%, 98%, 99%, 99.5% or 99.9% identical to SEQ ID NO:1 or 2. Preferably, the sequence identity is to SEQ ID NO:2. Preferably, the bacterial strain for use in the invention has the 16S rRNA gene sequence represented by SEQ ID NO:2. Most preferably, the bacterial strain is the strain deposited under accession number NCIMB 42787.

In particularly preferred embodiments, the invention provides a composition comprising a bacterial strain of the species Megasphaera massiliensis, for use in a method of treating or preventing depression.

Preferably, the bacterial strain of the genus Megasphaera for use in the invention produces one or more of the metabolites butyrate, valeric acid and hexanoic acid. Preferably, the bacterial strain of the genus Megasphaera for use in the invention consumes one or both of acetate and propionate. In preferred embodiments, the bacterial strain of the invention produces butyrate, valeric acid and hexanoic acid and consumes acetate and propionate. In particularly preferred embodiments, the invention provides a composition comprising a bacterial strain of the genus Megasphaera, most preferably of the species Megasphaera massiliensis, that produces butyrate, valeric acid and hexanoic acid and consumes acetate and propionate, for use in a method of treating or preventing depression.

In certain embodiments, the composition of the invention is for oral administration. Oral administration of the strains of the invention can be effective for psychiatric disorders. Also, oral administration is convenient for patients and practitioners and allows delivery to and/or partial or total colonisation of the intestine.

In certain embodiments, the composition of the invention comprises one or more pharmaceutically acceptable excipients or carriers.

In certain embodiments, the composition of the invention comprises a bacterial strain that has been lyophilised. Lyophilisation is an effective and convenient technique for preparing stable compositions that allow delivery of bacteria.

In certain embodiments, the invention provides a food product comprising the composition as described above.

Additionally, the invention provides a method of treating or preventing disorders characterised by serotonin deficiency, comprising administering a composition comprising a bacterial strain of the genus Megasphaera.

In developing the above invention, the inventors have identified and characterised a bacterial strain that is particularly useful for therapy. The Megasphaera massiliensis strain of the invention is shown to be effective for treating the diseases described herein, such as psychiatric disorders characterised by serotonin deficiency. Therefore, in another aspect, the invention provides a cell of the Megasphaera massiliensis strain deposited under accession number NCIMB 42787, or a derivative thereof. The invention also provides compositions comprising such cells, or biologically pure cultures of such cells. The invention also provides a cell of the Megasphaera massiliensis strain deposited under accession number NCIMB 42787, or a derivative thereof, for use in therapy, in particular for the diseases described herein.

### EMBODIMENTS OF THE INVENTION

1. A composition comprising a bacterial strain of the genus Megasphaera, for use in the treatment or prevention of a disorder characterised by serotonin deficiency.

2. The composition for use according to embodiment 1, in the treatment or prevention of a psychiatric disorder characterised by serotonin deficiency.

3. The composition for use according to embodiment 1 or 2, wherein the disorder is selected from the group consisting of: depression, anxiety, post-traumatic stress disorder (PTSD) and obsessive compulsive disorder.

4. The composition for use according to embodiment 3, wherein the disorder is depression.

5. The composition for use according to embodiment 3, wherein the disorder is PTSD.

6. The composition for use according to any preceding embodiment, wherein, when administered to a subject, the composition increases the production of serotonin in the subject via the tryptophan hydroxylase pathway.

7. The composition for use according to embodiment 6, wherein, the composition increases the production of serotonin in the brain of the subject.

8. The composition for use according to either of embodiments 6 or 7, wherein, when administered to the subject, the composition increases the expression of tryptophan hydroxylase 1.

9. The composition for use according to either of embodiments 6 or 7, wherein, when administered to the subject, the composition increases the expression of tryptophan hydroxylase 2.

10. The composition for use according to either of embodiments 6 or 7, wherein, when administered to the subject, the composition increases the expression of tryptophan hydroxylase 1 and 2.

11. The composition for use according to any preceding embodiment, wherein, when administered to a subject, the composition increases the expression of serotonin transporter in neurons of the subject.

12. The composition for use according to any preceding embodiment, wherein, when administered to a subject, the composition increases the secretion of serotonin in the subject, optionally in the brain of the subject.

13. The composition for use according to any preceding embodiment, wherein the bacterial strain produces one or more of the following metabolites: butyrate, valeric acid and hexanoic acid.

14. The composition for use according to any preceding embodiment, wherein the bacterial strain consumes one or both of acetate and propionate.

15. The composition for use according to any preceding embodiment, wherein the bacterial strain produces butyrate, valeric acid and hexanoic acid and consumes acetate and propionate.

16. The composition for use according to any preceding embodiment, wherein the bacterial strain has a 16S rRNA sequence that is at least 95%, 96%, 97%, 98%, 99%, 99.5% or 99.9% identical to the 16S rRNA sequence of a bacterial strain of the genus Megasphaera.

17. The composition for use according to any preceding embodiment, wherein the bacterial strain has a 16s rRNA gene sequence that is at least 95%, 96%, 97%, 98%, 99%, 99.5% or 99.9% identical to any one of SEQ ID NOs:11, 12, 13, 14, 15, or 16 or wherein the bacterial strain has a 16s rRNA gene sequence represented by any one of SEQ ID NOs:11, 12, 13, 14, 15 or 16.

18. The composition for use according to any preceding embodiment, wherein the bacterial strain is of Megasphaera massiliensis.

19. The composition for use according to any preceding embodiment, wherein the bacterial strain has a 16s rRNA gene sequence that is at least 95%, 96%, 97%, 98%, 99%, 99.5% or 99.9% identical to the 16s rRNA gene sequence of a bacterial strain of Megasphaera massiliensis.

20. The composition for use according to any preceding embodiment, wherein the bacterial strain has a 16s rRNA gene sequence that is at least 95%, 96%, 97%, 98%, 99%, 99.5% or 99.9% identical to SEQ ID NO:1 or 2.

21. The composition for use according to any preceding embodiment, wherein the bacterial strain has a 16s rRNA gene sequence that is at least 95%, 96%, 97%, 98%, 99%, 99.5% or 99.9% identical to SEQ ID NO:2, or wherein the bacterial strain has the 16s rRNA gene sequence represented by SEQ ID NO:2.

22. The composition for use according to any preceding embodiment, wherein the composition is for oral administration.

23. The composition for use according to any preceding embodiment, wherein the composition comprises one or more pharmaceutically acceptable excipients or carriers.

24. The composition for use according to any preceding embodiment, wherein the bacterial strain is lyophilised.

25. The composition for use according to any preceding embodiment, wherein the composition does not contain bacteria from any other genus or which comprises only de minimis or biologically irrelevant amounts of bacteria from another genus.

26. The composition for use according to embodiment 11, wherein the composition does not contain bacteria from any other species or which comprises only de minimis or biologically irrelevant amounts of bacteria from another species.

27. The composition for use according to any preceding embodiment, wherein the bacterial strain is viable and capable of partially or totally colonising the intestine.

28. The composition for use according to any preceding embodiment, wherein the composition comprises a single strain of the genus Megasphaera, or a single strain of the species Megasphaera massiliensis.

29. The composition for use according to any of embodiments 1-19 or 22, which comprises the Megasphaera bacterial strain as part of a microbial consortium.

30. A food product comprising the composition of any preceding embodiment, for the use of any preceding embodiment.

31. A vaccine composition comprising the composition of any preceding embodiment, for the use of any preceding embodiment.

32. A method of treating or preventing a disorder characterised by serotonin deficiency, comprising administering a composition comprising a bacterial strain of the genus Megasphaera to a subject in need thereof.

33. A cell of the Megasphaera massiliensis strain deposited under accession number NCIMB 42787, or a derivative thereof.

34. A composition comprising the cell of embodiment 28.

35. The composition of embodiment 29, comprising a pharmaceutically acceptable carrier or excipient.

36. A biologically pure culture of the Megasphaera massiliensis strain deposited under accession number NCIMB 42787, or a derivative thereof.

37. A cell of the Megasphaera massiliensis strain deposited under accession number NCIMB 42787, or a derivative thereof, for use in therapy.

38. The cell of embodiment 32, wherein the cell is for use according to any of embodiments 1-24.

39. The composition for use according to any preceding embodiment, wherein the subject to be treated has been administered or is to be administered a selective serotonin reuptake inhibitor.

40. A selective serotonin reuptake inhibitor for use in combination with a composition according to any of embodiments 1-24 in the treatment or prevention of a disorder characterised by any of embodiments 1-24.

41. A selective serotonin reuptake inhibitor for use in the treatment or prevention of a disorder characterised by serotonin deficiency, wherein the subject to be treated has received or is to receive a composition characterised by any of embodiments 1-24, 25 or 30.

42. The selective serotonin reuptake inhibitor of any of embodiments 34-36, wherein the selective serotonin reuptake inhibitor is selected from the list consisting of: citalopram, dapoxetine, escitalopram, fluoxetine, fluvoxamine, paroxetine, sertraline and vortioxetine.

43. A bacterial strain for use in therapy, wherein the bacterial strain has a 16S rRNA sequence that is at least 95%, 96%, 97%, 98%, 99%, 99.5% or 99.9% identical to any one of SEQ ID NOs:11, 12, 13, 14, 15 or 16.

44. A bacterial strain having the 16S rRNA sequence represented by any one of SEQ ID NOs: 11, 12, 13, 14, 15, or 16 for use in therapy.

### BRIEF DESCRIPTION OF DRAWINGS

The invention is illustrated by reference to the following Figures, in which:

FIG. 1 shows the levels of neurotransmitter metabolites in the brain following administration of NCIMB 42787.

FIG. 2 shows the fold-change in expression of tryptophan hydroxylase 1 and 2 (TPH1 and TPH2) in SH-SY5Y neuroblastoma cells. Cells were incubated with 10% NCIMB 42787 bacterial cell-free supernatant for 24 h.

FIG. 3 shows the fold-change in expression of tryptophan hydroxylase 1 and 2 (TPH1 and TPH2) in SH-SY5Y neuroblastoma cells. Cells were incubated with 5% NCIMB 42787 bacterial cell-free supernatant for 72 h.

FIG. 4 shows the fold-change in expression of SLC6A4 in SH-SY5Y neuroblastoma cells. Cells were incubated with 10% NCIMB 42787 bacterial cell-free supernatant for 24 h.

FIG. 5 shows the fold-change in expression of SLC6A4 in SH-SY5Y neuroblastoma cells. Cells were incubated with 5% NCIMB 42787 bacterial cell-free supernatant for 72 h.

FIG. 6 shows the fold-change in expression of tryptophan hydroxylase 1 (TPH1) in differentiated Caco2 cells. Cells were incubated with 10% NCIMB 42787 bacterial cell-free supernatant for 24 h.

FIG. 7 shows the fold-change in expression of SLC6A4 in differentiated Caco2 cells. Cells were incubated with 10% NCIMB 42787 bacterial cell-free supernatant for 24 h.

FIG. 8 shows the levels of metabolite production—organic acids in the supernatant

FIGS. 9A and 9B show the levels of metabolite production—Strains Ref 1, Ref 2 and Ref 3.

FIG. 10: Organic acid production and consumption by NCIMB 42787, NCIMB 43385, NCIMB 43388 and NCIMB 43389.

FIG. 11: NCIMB 42787 affects colonic Tph1 mRNA expression in BALB/c mice.

FIG. 12: Megasphaera reference strain NCIMB 43385 modulates mineralcorticoid receptor expression in BALB/c mice.

FIG. 13: Megapsphaera reference strain NCIMB 43385 modulates GABA A2 expression in the hippocampus and amygdala.

FIG. 14: Megasphaera reference strain NCIMB 43387 modulates expression of oxytocin receptor.

### DISCLOSURE OF THE INVENTION

Bacterial Strains

The compositions of the invention comprise a bacterial strain of the genus Megasphaera. The examples demonstrate that bacteria of this genus may be particularly useful for treating or preventing disorders characterised by serotonin deficiency. Preferably, the bacterial strain for use in the invention has a 16S rRNA gene sequence that is at least 95%, 96%, 97%, 98%, 99%, 99.5% or 99.9% identical to SEQ ID NO:1 or 2. Preferably, the sequence identity is to SEQ ID NO:2. Preferably, the bacterial strain for use in the invention has the 16S rRNA gene sequence represented by SEQ ID NO:2. The preferred bacterial strains are of the species Megasphaera massiliensis.

Examples of Megasphaera species for use in the invention include Megasphaera elsdenii, Megasphaera cerevisiae, Megasphaera massiliensis, Megasphaera indica, Megasphaera paucivorans, Megasphaera sueciensis and Megasphaera micronuciformis. A further example of a Megasphaera species for use in the invention is Megasphaera hexanoica. The Megasphaera are obligately anaerobic, lactate-fermenting, gastrointestinal microbe of ruminant and non-ruminant mammals, including humans.

The type strain of M. massiliensis is NP3 (=CSUR P245=DSM 26228)[17]. The GenBank accession number for the 16S rRNA gene sequences of M massiliensis strain NP3 is JX424772.1 (disclosed herein as SEQ ID NO: 1).

The Megasphaera massiliensis bacterium tested in the Examples is referred to herein as strain NCIMB 42787. A 16S rRNA gene sequence for the NCIMB 42787 strain that was tested is provided in SEQ ID NO:2.

Strain NCIMB 42787 was deposited with the international depositary authority NCIMB, Ltd. (Ferguson Building, Aberdeen, AB21 9YA, Scotland) by 4D Pharma Research Ltd. (Life Sciences Innovation Building, Cornhill Road, Aberdeen, AB25 2ZS, Scotland) on 13 Jul. 2017 and was assigned accession number NCIMB 42787.

Bacterial strains closely related to the strain tested in the examples are also expected to be effective for treating or preventing disorders characterised by serotonin deficiency. In certain embodiments, the bacterial strain for use in the invention has a 16S rRNA gene sequence that is at least 95%, 96%, 97%, 98%, 99%, 99.5% or 99.9% identical to the 16S rRNA gene sequence of a bacterial strain of Megasphaera massiliensis. Preferably, the bacterial strain for use in the invention has a 16S rRNA gene sequence that is at least 95%, 96%, 97%, 98%, 99%, 99.5% or 99.9% identical to SEQ ID NO:1 or 2. Preferably, the sequence identity is to SEQ ID NO:2. Preferably, the bacterial strain for use in the invention has the 16S rRNA gene sequence represented by SEQ ID NO:2.

Bacterial strains that are biotypes of strains NCIMB 42787 or NP3 are also expected to be effective for treating or preventing disorders characterised by serotonin deficiency. A biotype is a closely related strain that has the same or very similar physiological and biochemical characteristics.

Strains that are biotypes of strains NCIMB 42787 or NP3 and that are suitable for use in the invention may be identified by sequencing other nucleotide sequences for strains NCIMB 42787 or NP3. For example, substantially the whole genome may be sequenced and a biotype strain for use in the invention may have at least 95%, 96%, 97%, 98%, 99%, 99.5% or 99.9% sequence identity across at least 80% of its whole genome (e.g. across at least 85%, 90%, 95% or 99%, or across its whole genome). Other suitable sequences for use in identifying biotype strains may include hsp60 or repetitive sequences such as BOX, ERIC, (GTG)₅, or REP or [18]. Biotype strains may have sequences with at least 95%, 96%, 97%, 98%, 99%, 99.5% or 99.9% sequence identity to the corresponding sequence of the strains NCIMB 42787 or NP3.

Alternatively, strains that are biotypes of strains NCIMB 42787 or NP3 and that are suitable for use in the invention may be identified by using strains NCIMB 42787 or NP3 and restriction fragment analysis and/or PCR analysis, for example by using fluorescent amplified fragment length polymorphism (FAFLP) and repetitive DNA element (rep)-PCR fingerprinting, or protein profiling, or partial 16S or 23S rDNA sequencing. In preferred embodiments, such techniques may be used to identify other Megasphaera massiliensis strains.

In certain embodiments, strains that are biotypes of strains NCIMB 42787 or NP3 and that are suitable for use in the invention are strains that provide the same pattern as strains NCIMB 42787 or NP3 when analysed by amplified ribosomal DNA restriction analysis (ARDRA), for example when using Sau3AI restriction enzyme (for exemplary methods and guidance see, for example,[19]). Alternatively, biotype strains are identified as strains that have the same carbohydrate fermentation patterns as strains NCIMB 42787 or NP3.

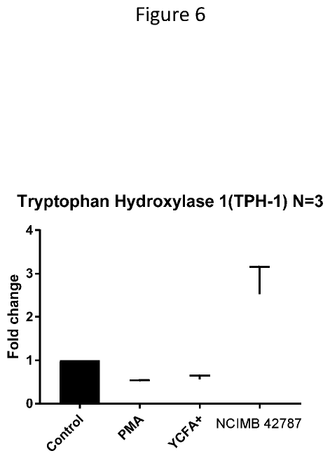

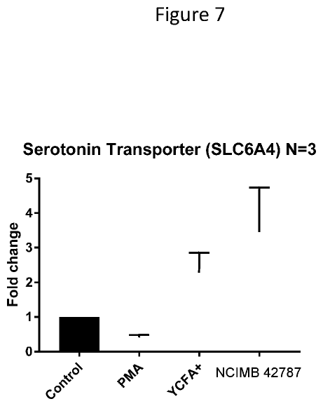

Bacterial strains that have similar growth patterns, metabolic type and/or surface antigens to strains NCIMB 42787 or NP3 may be useful in the invention. A useful strain will have comparable psychobiotic properties compared to strains NCIMB 42787 or NP3. In particular, a biotype strain will elicit comparable effects on the models shown in the Examples, which may be identified by using the culturing and administration protocols described in the Examples. In some embodiments, bacterial strains useful in the invention may be identified by routinely profiling the production and consumption of metabolites by a bacterial strain. The inventors have found that the bacterial strain used in the Examples produces butyrate, valeric acid and hexanoic acid and consumes acetate and propionate (see FIG. 6). The Megasphaera massiliensis strains Ref 1, Ref 2 and Ref 3 were also found to consume and produce these metabolites (see FIG. 7). Therefore, in some embodiments, the bacterial strain of the invention produces one or more of the metabolites butyrate, valeric acid and hexanoic acid. In some embodiments, the bacterial strain of the invention consumes one or both of acetate and propionate. In preferred embodiments, the bacterial strain of the invention produces butyrate, valeric acid and hexanoic acid and consumes acetate and propionate.

Other Megasphaera strains that are useful in the compositions and methods of the invention, such as biotypes of strains NCIMB 42787 or NP3, may be identified using any appropriate method or strategy, including the assays described in the examples. For instance, strains for use in the invention may be identified by culturing with SH-SY5Y neuroblastoma cells and then assessing the level of expression of tryptophan hydroxylase 1 and/or 2 and/or serotonin transporter. Strains for use in the invention may also be identified by feeding to mice and ex vivo screening brain tissue for increased secretion of serotonin, relative to controls.

A particularly preferred strain of the invention is the Megasphaera massiliensis NCIMB 42787 strain. This is the exemplary strain tested in the examples and shown to be effective for increasing expression of serotonin transporter and tryptophan hydroxylase1 and 2 and increasing secretion of serotonin. Therefore, the invention provides a cell, such as an isolated cell, of the Megasphaera massiliensis strain NCIMB 42787, or a derivative thereof. The invention also provides a composition comprising a cell of the Megasphaera massiliensis strain NCIMB 42787, or a derivative thereof. The invention also provides a biologically pure culture of the Megasphaera massiliensis strain NCIMB 42787. The invention also provides a cell of the Megasphaera massiliensis strain NCIMB 42787, or a derivative thereof, for use in therapy, in particular for the diseases described herein.

A particularly preferred strain of the invention is the Megasphaera massiliensis strain deposited under accession number NCIMB 42787. This is the exemplary NCIMB 42787 strain tested in the examples. Therefore, the invention provides a cell, such as an isolated cell, of the Megasphaera massiliensis strain deposited under accession number NCIMB 42787, or a derivative thereof. The invention also provides a composition comprising a cell of the Megasphaera massiliensis strain deposited under accession number NCIMB 42787, or a derivative thereof. The invention also provides a biologically pure culture of the Megasphaera massiliensis strain deposited under accession number NCIMB 42787. The invention also provides a cell of the Megasphaera massiliensis strain deposited under accession number NCIMB 42787, or a derivative thereof, for use in therapy, in particular for the diseases described herein.

A derivative of the strain of the invention may be a daughter strain (progeny) or a strain cultured (subcloned) from the original. A derivative of a strain of the invention may be modified, for example at the genetic level, without ablating the biological activity. In particular, a derivative strain of the invention is therapeutically active. A derivative strain will have comparable therapeutic activity to the NCIMB 42787 strain. In particular, a derivative strain will elicit comparable effects the level of expression of tryptophan hydroxylase 1 and/or 2 and/or serotonin transporter in neurons and comparable effects on the secretion of serotonin to the effects shown in the Examples, which may be identified by using the culturing and administration protocols described in the Examples. A derivative of the NCIMB 42787 strain will generally be a biotype of the NCIMB 42787 strain.

References to cells of the Megasphaera massiliensis NCIMB 42787 strain encompass any cells that have the same safety and therapeutic efficacy characteristics as the strain NCIMB 42787, and such cells are encompassed by the invention.

In preferred embodiments, the bacterial strains in the compositions of the invention are viable and capable of partially or totally colonising the intestine.

In certain embodiments, the composition of the invention does not comprise a cell of the Megasphaera massiliensis strain deposited under accession number NCIMB 42787.

The Examples further demonstrate other bacterial strains that are useful for treating or preventing disorders characterised by serotonin deficiency. These bacterial strains were deposited with the international depositary authority NCIMB, Ltd. (Ferguson Building, Aberdeen, AB21 9YA, Scotland) by 4D Pharma Research Ltd. (Life Sciences Innovation Building, Cornhill Road, Aberdeen, AB25 2ZS, Scotland) on 6 May 2019 as Megasphaera massiliensis (under accession numbers NCIMB 43388 and NCIMB 43389) and Megasphaera spp. (accession numbers NCIMB 43385, NCIMB 43386 and NCIMB 43387). Accordingly, in an alternative embodiment, the compositions of the invention comprise one or more of these bacterial strains, or biotypes or derivatives thereof. For the avoidance of doubt, Ref 1 referred to above is the strain deposited under accession number NCIMB 43385, Ref 2 referred to above is the strain deposited under accession number NCIMB 43388, and Ref 3 referred to above is the strain deposited under accession number NCIMB 43389.

Bacterial strains closely related to the strains tested in the Examples are also expected to be effective for treating or preventing disorders characterised by serotonin deficiency.

In certain embodiments, the bacterial strain for use in the invention is the Megasphaera massiliensis strain deposited under accession number NCIMB 43388. In certain embodiments, the invention provides a cell of the strain deposited under accession number NCIMB 43388, or a derivative thereof, for use in therapy. In certain embodiments, the invention provides a cell of the strain deposited under accession number NCIMB 43388, or derivative thereof for use in treating or preventing disorders characterised by serotonin deficiency. In certain embodiments, the invention provides a cell of the strain deposited under accession number NCIMB 43388, for use in any one of the diseases described herein.

In preferred embodiments, the invention provides a composition comprising the strain deposited at NCIMB under accession number NCIMB 43388, or a derivative or biotype thereof, preferably for use in the treatment or prevention of disorders characterised by serotonin deficiency.

In certain embodiments, the composition of the invention does not comprise a cell of the Megasphaera massiliensis strain deposited under accession number NCIMB 43388. In some embodiments, the bacterial strain in the compositions of the invention is a bacterial strain of the genus Megasphaera, wherein the bacterial strain is not the strain deposited under accession number NCIMB 43388. In some embodiments, the bacterial strain in the compositions of the invention is a bacterial strain of the species Megasphaera massiliensis, wherein the bacterial strain is not the strain deposited under accession number NCIMB 43388.

Accordingly, in certain embodiments, the bacterial strain for use in the invention is the Megasphaera massiliensis strain deposited under accession number NCIMB 43389. In certain embodiments, the invention provides a cell of the strain deposited under accession number NCIMB 43389, or a derivative thereof, for use in therapy. In certain embodiments, the invention provides a cell of the strain deposited under accession number NCIMB 43389, or derivative thereof for use in treating or preventing disorders characterised by serotonin deficiency. In certain embodiments, the invention provides a cell of the strain deposited under accession number NCIMB 43389, for use in any one of the diseases described herein.

In preferred embodiments, the invention provides a composition comprising the strain deposited at NCIMB under accession number NCIMB 43389, or a derivative or biotype thereof, preferably for use in the treatment or prevention of disorders characterised by serotonin deficiency.

In certain embodiments, the composition of the invention does not comprise a cell of the Megasphaera massiliensis strain deposited under accession number NCIMB 43389. In some embodiments, the bacterial strain in the compositions of the invention is a bacterial strain of the genus Megasphaera, wherein the bacterial strain is not the strain deposited under accession number NCIMB 43389. In some embodiments, the bacterial strain in the compositions of the invention is a bacterial strain of the species Megasphaera massiliensis, wherein the bacterial strain is not the strain deposited under accession number NCIMB 43389.

In certain embodiments, the bacterial strain for use in the invention has a 16S rRNA sequence that is at least 95%, 96%, 97%, 98%, 99%, 99.5% or 99.9% identical to SEQ ID NO:12. In certain embodiments, the bacterial strain for use in the invention has the 16S rRNA sequence represented by SEQ ID NO: 12. In certain embodiments, the invention provides a bacterial strain having a 16S rRNA sequence that is at least 95%, 96%, 97%, 98%, 99%, 99.5% or 99.9% identical to SEQ ID NO:12 for use in therapy. In certain embodiments, the invention provides a bacterial strain having the 16S rRNA sequence represented by SEQ ID NO:12 for use in therapy.

In certain embodiments, the bacterial strain for use in the invention has a 16S rRNA sequence that is at least 95%, 96%, 97%, 98%, 99%, 99.5% or 99.9% identical to SEQ ID NO:13. In certain embodiments, the bacterial strain for use in the invention has the 16S rRNA sequence represented by SEQ ID NO: 13. In certain embodiments, the invention provides a bacterial strain having a 16S rRNA sequence that is at least 95%, 96%, 97%, 98%, 99%, 99.5% or 99.9% identical to SEQ ID NO:13 for use in therapy. In certain embodiments, the invention provides a bacterial strain having the 16S rRNA sequence represented by SEQ ID NO:13 for use in therapy.

In certain embodiments, the bacterial strain for use in the invention has a 16S rRNA sequence that is at least 95%, 96%, 97%, 98%, 99%, 99.5% or 99.9% identical to the 16S rRNA sequence of a bacterial strain of the genus Megasphaera. In certain embodiments, the bacterial strain for use in the invention is of the genus Megasphaera.

In certain embodiments, the bacterial strain for use in the invention is the Megasphaera strain deposited under accession number NCIMB 43385. In certain embodiments, the invention provides a cell of the strain deposited under accession number NCIMB 43385, or a derivative thereof, for use in therapy. In certain embodiments, the invention provides a cell of the strain deposited under accession number NCIMB 43385, or derivative thereof for use in treating or preventing disorders characterised by serotonin deficiency. In certain embodiments, the invention provides a cell of the strain deposited under accession number NCIMB 43385, for use in any one of the diseases described herein.

In preferred embodiments, the invention provides a composition comprising the strain deposited at NCIMB under accession number NCIMB 43385, or a derivative or biotype thereof, preferably for use in the treatment or prevention of disorders characterised by serotonin deficiency.

In certain embodiments, the composition of the invention does not comprise a cell of the Megasphaera massiliensis strain deposited under accession number NCIMB 43385. In some embodiments, the bacterial strain in the compositions of the invention is a bacterial strain of the genus Megasphaera, wherein the bacterial strain is not the strain deposited under accession number NCIMB 43385. In some embodiments, the bacterial strain in the compositions of the invention is a bacterial strain of the species Megasphaera massiliensis, wherein the bacterial strain is not the strain deposited under accession number NCIMB 43385.

In certain embodiments, the bacterial strain for use in the invention is the Megasphaera strain deposited under accession number NCIMB 43386. In certain embodiments, the invention provides a cell of the strain deposited under accession number NCIMB 43386, or a derivative thereof, for use in therapy. In certain embodiments, the invention provides a cell of the strain deposited under accession number NCIMB 43386, or derivative thereof for use in treating or preventing disorders characterised by serotonin deficiency. In certain embodiments, the invention provides a cell of the strain deposited under accession number NCIMB 43386, for use in any one of the diseases described herein.

In preferred embodiments, the invention provides a composition comprising the strain deposited at NCIMB under accession number NCIMB 43386, or a derivative or biotype thereof, preferably for use in the treatment or prevention of disorders characterised by serotonin deficiency.

In certain embodiments, the composition of the invention does not comprise a cell of the Megasphaera massiliensis strain deposited under accession number NCIMB 43386. In some embodiments, the bacterial strain in the compositions of the invention is a bacterial strain of the genus Megasphaera, wherein the bacterial strain is not the strain deposited under accession number NCIMB 43386. In some embodiments, the bacterial strain in the compositions of the invention is a bacterial strain of the species Megasphaera massiliensis, wherein the bacterial strain is not the strain deposited under accession number NCIMB 43386.

In certain embodiments, the bacterial strain for use in the invention is the Megasphaera strain deposited under accession number NCIMB 43387. In certain embodiments, the invention provides a cell of the strain deposited under accession number NCIMB 43387, or a derivative thereof, for use in therapy. In certain embodiments, the invention provides a cell of the strain deposited under accession number NCIMB 43387, or derivative thereof for use in treating or preventing disorders characterised by serotonin deficiency. In certain embodiments, the invention provides a cell of the strain deposited under accession number NCIMB 43387, for use in any one of the diseases described herein.

In preferred embodiments, the invention provides a composition comprising the strain deposited at NCIMB under accession number NCIMB 43387, or a derivative or biotype thereof, preferably for use in the treatment or prevention of disorders characterised by serotonin deficiency.

In certain embodiments, the composition of the invention does not comprise a cell of the Megasphaera massiliensis strain deposited under accession number NCIMB 43387. In some embodiments, the bacterial strain in the compositions of the invention is a bacterial strain of the genus Megasphaera, wherein the bacterial strain is not the strain deposited under accession number NCIMB 43387. In some embodiments, the bacterial strain in the compositions of the invention is a bacterial strain of the species Megasphaera massiliensis, wherein the bacterial strain is not the strain deposited under accession number NCIMB 43387.

In certain embodiments, the bacterial strain for use in the invention has a 16S rRNA sequence that is at least 95%, 96%, 97%, 98%, 99%, 99.5% or 99.9% identical to SEQ ID NO:11. In certain embodiments, the bacterial strain for use in the invention has a 16S rRNA sequence that is at least 95%, 96%, 97%, 98%, 99%, 99.5% or 99.9% identical to SEQ ID NO: 14. In certain embodiments, the bacterial strain for use in the invention has a 16S rRNA sequence that is at least 95%, 96%, 97%, 98%, 99%, 99.5% or 99.9% identical to SEQ ID NO: 15. In certain embodiments, the bacterial strain for use in the invention has a 16S rRNA sequence that is at least 95%, 96%, 97%, 98%, 99%, 99.5% or 99.9% identical to SEQ ID NOs:11, 14 or 15. In certain embodiments, the invention provides a bacterial strain having a 16S rRNA sequence that is at least 95%, 96%, 97%, 98%, 99%, 99.5% or 99.9% identical to SEQ ID NOs: 11, 14 or 15 for use in therapy.

In certain embodiments, the bacterial strain for use in the invention has the 16S rRNA sequence represented by SEQ ID NO: 11. In certain embodiments, the bacterial strain for use in the invention has the 16S rRNA sequence represented by SEQ ID NO: 14. In certain embodiments, the bacterial strain for use in the invention has the 16S rRNA sequence represented by SEQ ID NO: 15. In certain embodiments, the bacterial strain for use in the invention has the 16S rRNA sequence represented by SEQ ID NOs: 11, 14 or 15. In certain embodiments, the invention provides a bacterial strain having the 16S rRNA sequence represented by SEQ ID NOs: 11, 14 or 15 for use in therapy.

Bacterial strains that are biotypes of one or more of the strains deposited under accession numbers NCIMB 43385, NCIMB 43386, NCIMB 43387, NCIMB 43388 and/or NCIMB 43389 are also expected to be effective for treating or preventing disorders characterised by serotonin deficiency. A biotype is a closely related strain that has the same or very similar physiological and biochemical characteristics.

In certain embodiments, the invention provides the bacterial strains deposited under accession numbers NCIMB 43385, NCIMB 43386, NCIMB 43387, NCIMB 43388 and/or NCIMB 43389, or biotypes thereof, for use in therapy.

Strains that are biotypes of one or more of the deposited under accession numbers NCIMB 43385, NCIMB 43386, NCIMB 43387, NCIMB 43388 and/or NCIMB 43389 and that are suitable for use in the invention may be identified by sequencing other nucleotide sequences for one or more of the strains deposited under accession numbers NCIMB 43385, NCIMB 43386, NCIMB 43387, NCIMB 43388 and/or NCIMB 43389. For example, substantially the whole genome may be sequenced and a biotype strain for use in the invention may have at least 85%, 90%, 95%, 96%, 97%, 98%, 99%, 99.5% or 99.9% sequence identity across at least 80% of its whole genome (e.g. across at least 85%, 90%, 95% or 99%, or across its whole genome). Other suitable sequences for use in identifying biotype strains may include hsp60 or repetitive sequences such as BOX, ERIC, (GTG)₅, or REP. Biotype strains may have sequences with at least 85%, 90%, 95%, 96%, 97%, 98%, 99%, 99.5% or 99.9% sequence identity to the corresponding sequence of one or more of the strains deposited under accession numbers NCIMB 43385, NCIMB 43386, NCIMB 43387, NCIMB 43388 and/or NCIMB 43389.

Alternatively, strains that are biotypes of one or more of the strains deposited under accession numbers NCIMB 43385, NCIMB 43386, NCIMB 43387, NCIMB 43388 and/or NCIMB 43389 and that are suitable for use in the invention may be identified by using one or more of the strains deposited under accession numbers NCIMB 43385, NCIMB 43386, NCIMB 43387, NCIMB 43388 and/or NCIMB 43389 and restriction fragment analysis and/or PCR analysis, for example by using fluorescent amplified fragment length polymorphism (FAFLP) and repetitive DNA element (rep)-PCR fingerprinting, or protein profiling, or partial 16S or 23S rDNA sequencing. In preferred embodiments, such techniques may be used to identify other Megasphaera massiliensis strains.

In certain embodiments, strains that are biotypes of one or more of the strains deposited under accession numbers NCIMB 43385, NCIMB 43386, NCIMB 43387, NCIMB 43388 and/or NCIMB 43389 and that are suitable for use in the invention are strains that provide the same pattern as one or more of the strains deposited under accession numbers NCIMB 43385, NCIMB 43386, NCIMB 43387, NCIMB 43388 and/or NCIMB 43389 when analysed by amplified ribosomal DNA restriction analysis (ARDRA), for example when using Sau3AI restriction enzyme. Alternatively, biotype strains are identified as strains that have the same carbohydrate fermentation patterns as one or more of the strains deposited under accession numbers NCIMB 43385, NCIMB 43386, NCIMB 43387, NCIMB 43388 and/or NCIMB 43389.

Other strains that are useful in the compositions and methods of the invention, such as biotypes of one or more of the strains deposited under accession numbers NCIMB 43385, NCIMB 43386, NCIMB 43387, NCIMB 43388 and/or NCIMB 43389, may be identified using any appropriate method or strategy, including the assays described in the Examples. For instance, strains for use in the invention may be identified by culturing bacterial cell-free supernatant with SH-SY5Y neuroblastoma cells and then assessing the level of expression of tryptophan hydroxylase 1 and/or 2 and/or serotonin transporter. Strains for use in the invention may also be identified by feeding to mice and ex vivo screening brain tissue for increased secretion of serotonin, relative to controls.

In certain embodiments, preferred strains of the invention are strains deposited under accession numbers NCIMB 43385, NCIMB 43386, NCIMB 43387, NCIMB 43388 and/or NCIMB 43389. In certain embodiments, the invention provides a cell, such as an isolated cell, of one or more of the strains deposited under accession numbers NCIMB 43385, NCIMB 43386, NCIMB 43387, NCIMB 43388 and/or NCIMB 43389, or a derivative thereof. The invention also provides a composition comprising a cell of one of more of the strains deposited under accession numbers NCIMB 43385, NCIMB 43386, NCIMB 43387, NCIMB 43388 and/or NCIMB 43389, or a derivative thereof. The invention also provides a biologically pure culture of one or more of the strains deposited under accession numbers NCIMB 43385, NCIMB 43386, NCIMB 43387, NCIMB 43388 and/or NCIMB 43389. The invention also provides a cell of one or more of the strains deposited under accession numbers NCIMB 43385, NCIMB 43386, NCIMB 43387, NCIMB 43388 and/or NCIMB 43389, or a derivative thereof, for use in therapy, in particular for the diseases described herein.

A derivative of the strain of the invention may be a daughter strain (progeny) or a strain cultured (subcloned) from the original. A derivative of a strain of the invention may be modified, for example at the genetic level, without ablating the biological activity. In particular, a derivative strain of the invention is therapeutically active. A derivative strain will have comparable therapeutic activity to one or more of the strains deposited under accession numbers NCIMB 43385, NCIMB 43386, NCIMB 43387, NCIMB 43388 and/or NCIMB 43389. In particular, a derivative strain will elicit comparable effects the level of expression of tryptophan hydroxylase 1 and/or 2 and/or serotonin transporter in neurons and comparable effects on the secretion of serotonin to the effects shown in the Examples, which may be identified by using the culturing and administration protocols described in the Examples. A derivative of one or more of the strains deposited under accession numbers NCIMB 43385, NCIMB 43386, NCIMB 43387, NCIMB 43388 and/or NCIMB 43389 will generally be a biotype of one or more of the strains deposited under accession numbers NCIMB 43385, NCIMB 43386, NCIMB 43387, NCIMB 43388 and/or NCIMB 43389, respectively.

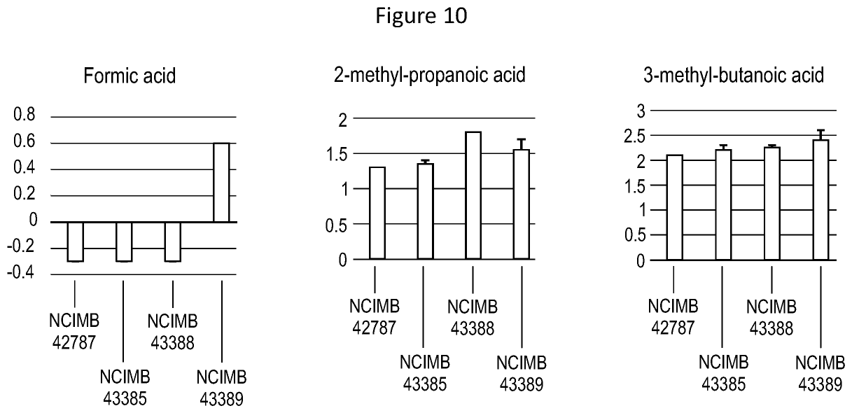

The inventors have found that the bacterial strain used in the Examples produces 2-methyl-propanoic acid and 3-methyl-propanoic acid and consumes formic acid (see FIG. 10). The strains deposited under accession numbers NCIMB 43385, NCIMB 43388 and NCIMB 43389 were also found to produce 2-methyl-propanoic acid and 3-methyl-propanoic acid. In addition, the strains deposited under accession numbers NCIMB 43385 and NCIMB 43388 were also found to consume formic acid. Therefore, in some embodiments, the bacterial strain of the invention produces one or more of the metabolites 2-methyl-propanoic acid and 3-methyl-propanoic acid. In some embodiments, the bacterial strain of the invention consumes formic acid. In some embodiments, the bacterial strain of the invention produces 2-methyl-propanoic acid and 3-methyl-propanoic acid and consumes formic acid. In preferred embodiments, the bacterial strain of the invention produces butyrate, valeric acid, hexanoic acid, 2-methyl-propanoic acid and 3-methyl-propanoic acid, and consumes acetate, propionate and formic acid.

In certain embodiments, the compositions of the invention do not comprise Megasphaera elsdenii. In certain embodiments, the bacterial strain useful in the compositions and methods of the invention is not Megasphaera elsdenii.

In certain embodiments, a strain for use in the invention is the Megasphaera elsdenii strain deposited under accession number NCIMB 8927. This is the exemplary M. elsdenii strain tested in the examples. Therefore, the invention provides a cell of the Megasphaera elsdenii strain deposited under accession number NCIMB 8927, or a derivative thereof, for use in treating or preventing disorders characterised by serotonin deficiency, in particular the diseases described herein. In certain embodiments, the invention provides bacterial strains that are biotypes or derivatives of the Megasphaera elsdenii strain deposited under NCIMB 8927 for use in treating or preventing disorders characterised by serotonin deficiency.

The GenBank accession number for the 16S rRNA gene sequence of the M elsdenii strain deposited under accession number NCIMB 8927 is LC036319.1 (disclosed herein as SEQ ID NO: 16). In certain embodiments, the bacterial strain for use in the invention has a 16S rRNA gene sequence that is at least 95%, 96%, 97%, 98%, 99%, 99.5% or 99.9% identical to the 16S rRNA gene sequence of a bacterial strain of Megasphaera elsdenii.

In certain embodiments, the bacterial strain for use in the invention has a 16S rRNA gene sequence that is at least 95%, 96%, 97%, 98%, 99%, 99.5% or 99.9% identical to SEQ ID NO:16. In certain embodiments, the bacterial strain for use in the invention has the 16S rRNA sequence represented by SEQ ID NO: 16. In certain embodiments, the invention provides a bacterial strain having a 16S rRNA sequence that is at least 95%, 96%, 97%, 98%, 99%, 99.5% or 99.9% identical to SEQ ID NO:16 for use in treating or preventing disorders characterised by serotonin deficiency. In certain embodiments, the invention provides a bacterial strain having the 16S rRNA sequence represented by SEQ ID NO: 16 for use in treating or preventing disorders characterised by serotonin deficiency.

Therapeutic Uses

As demonstrated in the examples, the bacterial compositions of the invention are effective for treating disorders characterised by serotonin deficiency. “Serotonin deficiency” refers to lower than optimal levels of serotonin in a subject that may lead to, amongst other symptoms, anxiety, worrying, panic, phobias, mental obsessions, behavioural compulsions, somnipathy, aggression, and/or appetite suppression or cravings. Generally, serotonin deficiency is diagnosed in a subject based on observing one or more of these symptoms in a subject as opposed to biochemically assaying serotonin levels in the subject. Therefore, “serotonin deficiency” describes a range of disorders in which serotonin deficiency may be a contributing factor to pathobiology. In some embodiments, “disorders characterised by serotonin deficiency” also comprises those in which increasing the concentration of serotonin in vivo has a beneficial effect in the treatment of a subject (for example, by alleviating symptoms associated with the disorder), wherein the subject does not exhibit low levels of serotonin (i.e. below base-line levels). In other words, disorders characterised by serotonin deficiency may include any disorder in which increasing the in vivo concentration of serotonin has a beneficial effect.

The inventors have found that treatment with compositions of the invention increase the expression of tryptophan hydroxylase, an enzyme involved in the production of serotonin. Tryptophan hydroxylase catalyses the hydroxylation of L-tryptophan during the formation of the serotonin precursor 5-hydroxytryptophan, which is the rate limiting step in the formation of serotonin. Therefore, the compositions of the invention may be useful for treating or preventing disorders characterised by serotonin deficiency by increasing the production of serotonin via the tryptophan hydroxylase pathway.

In preferred embodiments, the composition of the invention increases the production of serotonin via the tryptophan hydroxylase pathway in the brain.

More specifically, the inventors have found that the composition of the invention increases the expression of tryptophan hydroxylase 2 in neurons. Tryptophan hydroxylase 2 is the main isoform expressed in serotonergic neurons in the brain [20]. Therefore, in some embodiments, the composition of the invention may be useful for treating or preventing disorders characterised by serotonin deficiency by increasing the production of serotonin by increasing the expression of Tryptophan hydroxylase 2 in the subject, for example, in the brain of the subject.

The inventors have also found that the composition of the invention increases the expression of tryptophan hydroxylase 1 in neurons. Tryptophan hydroxylase 1 is predominantly expressed in tissues other than the brain. However, Tryptophan hydroxylase 1 expression has been shown to be increased in serotonergic neurons of the brain in subjects experiencing stress [21]. Without wishing to be bound by theory, the increased expression of Tryptophan hydroxylase 1 in serotonergic neurons of subjects experiencing stress may be a biological coping mechanism by enhancing the production of serotonin in subjects, thereby “self” treating or preventing stress-induced psychiatric disorders characterised by serotonin deficiency. This suggests that even though Tryptophan hydroxylase 1 is not the predominant isoform found in the region of the brain from which serotonin signalling predominantly takes place, the increased expression of Tryptophan hydroxylase 1 in the relevant brain region is effective in increasing the local concentration of serotonin at the active site. Therefore, increasing the expression of tryptophan hydroxylase 1 and/or 2 in neurons of a subject may be effective for treating or preventing disorders characterised by serotonin deficiency, in particular psychiatric disorders characterised by serotonin deficiency. In some embodiments, the composition of the invention may be useful for treating or preventing disorders characterised by serotonin deficiency by increasing the production of serotonin by increasing the expression of Tryptophan hydroxylase 1 in the subject, for example, in the brain of the subject. In some embodiments, the composition of the invention is for use in the treatment or prevention of a stress-induced disorder characterised by serotonin deficiency

Furthermore, treatment with the composition of the invention increases the expression of the serotonergic neuron marker serotonin transporter in neurons (SERT). Therefore, the composition of the invention may increase the differentiation of immature neurons to serotonergic neurons in vivo. Serotonergic neurons produce serotonin in vivo. Therefore the composition of the invention may be effective in the treatment or prevention of a disorder characterised by serotonin deficiency, by increasing the production of serotonin in vivo by increasing the number of serotonergic neurons in the subject.

Furthermore, the inventors have found that treatment with the composition of the invention increases the secretion of serotonin in the brain. Therefore, the composition of the invention may be particularly effective in the treatment or prevention of a disorder characterised by serotonin deficiency in a subject, by increasing the secretion of serotonin in the subject. In preferred embodiments, the composition of the invention increases the secretion of serotonin in the brain of the subject.

In certain embodiments, the compositions of the invention are for use in increasing the number of serotonergic neurons in the Raphe Nuclei.

In certain embodiments, the compositions of the invention are for use in increasing serotonin levels. In certain embodiments, the compositions of the invention are for use in increasing 5-hydroxytryptophan levels. In certain embodiments, the compositions of the invention are for use in increasing serotonin and 5-hydroxytryptohan levels. In certain embodiments, the serotonin and/or 5-hydroxytryptohan are increased in the striatum. Serotonin and 5-hydroxytryptophan levels may be measured using any appropriate method known in the art, such as an enzyme-linked immunosorbent assay (ELISA), for example in CSF (for example as described in [22]), or a reverse-phase HPLC method, perhaps with electrochemical detection, for example in plasma or CSF (for example as described in [23]).

In certain embodiments, the compositions of the invention are for use in treating a patient suspected of having or identified as having lowered serotonin levels.

Depression

The composition of the invention may be particularly useful in the treatment or prevention of depression. Depression is characterised by a state of low mood and aversion to activity and can affect a subject's thoughts, behaviour, tendencies, feelings and sense of well-being. The biological mechanisms underlying depression have not been fully characterised, but low levels of serotonin, i.e. serotonin deficiency, has been shown to at least contribute towards depression [24]. Therefore, the composition of the invention may be effective in the treatment of depression, for example, by increasing the bioavailability of serotonin.

Depression describes a variety of different low mood psychiatric disorders characterised by one or more of the following symptoms: low mood, sadness, hopelessness, helplessness, low self-esteem, tearfulness, irritability, lack of motivation, anxiety, loss or appetite, gain of appetite, weight loss, weight gain, constipation, loss of libido and somnipathy. The composition of the invention may be effective in the treatment of one or more of these symptoms associated with depression. Non-limiting examples of the sub-categories of depression for which the invention may be an effective treatment include: major depressive disorder, persistent depressive disorder, bipolar disorder, postpartum depression, premenstrual dysphoric disorder, seasonal affective disorder and atypical depression.

In some embodiments, the composition of the invention is for use in the treatment or prevention of depression in a subject, wherein, when administered to the subject the composition increases the expression of tryptophan hydroxylase in the subject. In some embodiments, when administered to the subject, the composition increases the expression of tryptophan hydroxylase 1 in the subject. In some embodiments, when administered to the subject, the composition increases the expression of tryptophan hydroxylase 2 in the subject. In some embodiments, when administered to the subject, the composition increases the expression of tryptophan hydroxylase 1 and 2 in the subject. In some embodiments, the composition increases the expression of tryptophan hydroxylase expression in neurons in the subject. In some embodiments, the composition increases the expression of tryptophan hydroxylase expression in serotonergic neurons in the subject. In some embodiments, the composition of the invention increases the expression of tryptophan hydroxylase in serotonergic neurons in the raphe nuclei of the subject. The raphe nuclei comprise a moderate-size cluster of nuclei found in the brain stem. One of the main functions of these nuclei is to release serotonin to the rest of the brain. Therefore, increasing the expression of tryptophan hydroxylase in neurons of the raphe nuclei may be effective in increasing the production of serotonin accessible to the brain.

In some embodiments, the composition of the invention is for use in the treatment or prevention of depression in a subject, wherein, when administered to the subject the composition increases the expression of serotonin transporter in the subject. In some embodiments, the composition increases the expression of serotonin transporter in neurons in the subject. In some embodiments, the composition increases the expression of serotonin transporter in serotonergic neurons in the subject. In some embodiments, the composition of the invention increases the expression of serotonin transporter in serotonergic neurons in the raphe nuclei of the subject.

In some embodiments, the composition of the invention is for use in the treatment or prevention of depression in a subject, wherein the subject has been diagnosed with depression. Depression may be diagnosed in a subject using the Hamilton Depression Rating Scale. Depression may also be diagnosed in a subject in accordance with criteria specified in the Diagnostic and Statistical Manual of Mental Disorders (DSM-5) [25]. A subject may be diagnosed with clinical depression in accordance with DSM-5 by the presence of five or more of the following depressive symptoms: 1) Depressed mood most of the day, nearly every day, as indicated by either subjective report (e.g., feeling sad, blue, “down in the dumps,” or empty) or observations made by others (e.g., appears tearful or about to cry). (In children and adolescents, this may present as an irritable or cranky, rather than sad, mood.); 2) markedly diminished interest or pleasure in all, or almost all, activities every day, such as no interest in hobbies, sports, or other things the person used to enjoy doing; 3) Significant weight loss when not dieting or weight gain (e.g., a change of more than 5 percent of body weight in a month), or decrease or increase in appetite nearly every day; 4) Insomnia (inability to get to sleep or difficulty staying asleep) or hypersomnia (sleeping too much) nearly every day; 5) more days than not, problems with sitting still, including constant restlessness, pacing, or picking at one's cloths (called psychomotor agitation by professionals); or the opposite, a slowing of one's movements, talking very quietly with slowed speech (called psychomotor retardation by professionals); 6) fatigue, tiredness, or loss of energy nearly every day—even the smallest tasks, like dressing or washing, seem difficult to do and take longer than usual; 7) Feelings of worthlessness or excessive or inappropriate guilt nearly every day (e.g., ruminating over minor past failings); 8) Diminished ability to think or concentrate, or indecisiveness, nearly every day (e.g., appears easily distracted, complains of memory difficulties); 9) Recurrent thoughts of death (notjust fear of dying), recurrent suicidal ideas without a specific plan, or a suicide attempt or a specific plan for committing suicide. In some embodiments, the composition of the invention is for use in the treatment of clinical depression.

In some embodiments, the composition of the invention is for use in the treatment or one or more of the above symptoms of depression. Treatment of one or more symptoms of depression can be determined by clinically assessing a subject pre and post-administration of the composition to ascertain an improvement in one or more symptoms associated with depression. In some embodiments, the composition of the invention improves the symptoms associated with depression according to a symptomatic or diagnostic test and/or scale. In certain embodiments, the test or scale is the Hamilton Depression Rating Scale (HAM-D 17; depression). In certain embodiments, the test or scale is that specified in the DSM-5.

In some embodiments, the compositions of the invention improve the Clinical Global Impression-Global Improvement (CGI-I) scale for assessing psychiatric and neurological disorders. In some embodiments, the compositions of the invention display a positive effect on global social and occupational impairment of the subject clinically diagnosed with depression.

In some embodiments, the composition is for use in the treatment of prevention of one or more symptoms of depression. For example, the composition of the invention may be for use in the treatment or prevention of one or more of psychological symptoms of depression selected from the list consisting of: low mood, sadness, hopelessness, helplessness, low self-esteem, tearfulness, irritability, lack of motivation and anxiety. In some embodiments, the composition is for use in the treatment of prevention of one or more physical symptoms of depression selected from the list consisting of: loss or appetite, gain of appetite, weight loss, weight gain, constipation, loss of libido and somnipathy.

In some embodiments, the composition of the invention is for use in combination with a selective serotonin reuptake inhibitor (SSRI). SSRIs are a type of drug that selectively block the reuptake of serotonin by blocking the activity of the serotonin transporter (SERT). SERT is a type of monoamine transporter protein that transports serotonin from the synaptic cleft to a presynaptic neuron. Therefore, SSRIs increase the residence time of serotonin in the synapse to increase the half-life of serotonin signalling events. The combination of an SSRI and the composition of the invention is particularly useful in the context of the treatment of depression since it may further improve the bioavailability of serotonin in a subject.

In some embodiments, the composition of the invention is for simultaneous, sequential or separate use with an SSRI in the treatment or prevention of depression.

In some embodiments, the invention relates to an SSRI for simultaneous, sequential or separate use with the composition of the invention in the treatment or prevention of depression.

In some embodiments, the invention relates to an SSRI for use in the treatment or prevention of depression in a subject, wherein the subject is to be administered or has been administered with the composition of the invention.

In some embodiments, the composition of the invention is for use in the treatment or prevention of depression in a subject, wherein the subject is to be administered or has been administered with an SSRI.

Exemplary SSRIs suitable for use in the invention include, but are not limited to citalopram, dapoxetine, escitalopram, fluoxetine, fluvoxamine, paroxetine, sertraline and vortioxetine.

In some embodiments, the composition of the invention is for use in the treatment of depression in combination with one or more anti-depressant therapies selected from the list consisting of: cognitive behavioural therapy, counselling, interpersonal therapy, psychodynamic therapy, tricyclic antidepressants, serotonin-noradrenaline reuptake inhibitors, noradrenaline and specific serotonergic antidepressants and monoamine oxidase inhibitors.

Anxiety

The composition of the invention may be particularly useful in the treatment or prevention of anxiety. Anxiety is characterised as a feeling or unease, such as worry or fear, which can be mild or severe. Anxiety describes a range of conditions associated with the above-described symptoms. Non-limiting examples of anxiety disorders that may be treated or prevented by the composition of the invention include: panic disorder, phobias, post-traumatic stress disorder, social anxiety disorder and general anxiety disorder. Studies have shown that increasing the bioavailability of serotonin in a subject may be effective in the treatment of anxiety [26].

In some embodiments, the composition of the invention is for use in the treatment or prevention of general anxiety disorder (GAD) in a subject. GAD is a long-term condition that causes a subject to feel anxious about a wide range of situations and issues, rather than one specific event. Subjects with GAD feel anxious most days: when anxiety associated with one particular thought is resolved, another may appear.

In some embodiments, the composition of the invention is for use in the treatment or prevention of GAD in a subject, wherein, when administered to the subject the composition increases the expression of tryptophan hydroxylase. In some embodiments, when administered to the subject, the composition increases the expression of tryptophan hydroxylase 1 in the subject. In some embodiments, when administered to the subject, the composition increases the expression of tryptophan hydroxylase 2 in the subject. In some embodiments, when administered to the subject, the composition increases the expression of tryptophan hydroxylase 1 and 2 in the subject. In some embodiments, the composition increases the expression of tryptophan hydroxylase expression in neurons. In some embodiments, the composition increases the expression of tryptophan hydroxylase expression in serotonergic neurons.

In some embodiments, the composition of the invention increases the expression of tryptophan hydroxylase in serotonergic neurons in the raphe nuclei of the subject. The raphe nuclei comprise a moderate-size cluster of nuclei found in the brain stem. One of the main functions of these nuclei is to release serotonin to the rest of the brain. Therefore, increasing the expression of tryptophan hydroxylase in neurons of the raphe nuclei is beneficial in increasing the production of serotonin accessible to the brain. In some embodiments, when administered to a subject, the composition increases the production of serotonin in the subject via the tryptophan hydroxylase pathway.

In some embodiments, the composition of the invention is for use in the treatment or prevention of GAD in a subject, wherein, when administered to the subject the composition increases the expression of serotonin transporter in the subject. In some embodiments, the composition increases the expression of serotonin transporter in neurons in the subject. In some embodiments, the composition increases the expression of serotonin transporter in serotonergic neurons in the subject. In some embodiments, the composition of the invention increases the expression of serotonin transporter in serotonergic neurons in the raphe nuclei of the subject.

In some embodiments, the composition of the invention is for use in the treatment or prevention of GAD in a subject, wherein the subject has been diagnosed with GAD. According to the DSM-5, a GAD diagnosis can be made if the subject displays the following symptoms: the presence of excessive anxiety and worry about a variety of topics, events, or activities, wherein the worry occurs more often than not for at least 6 months and is clearly excessive; the worry is experienced as very challenging to control; the anxiety and worry are accompanied with at least three of the following physical or cognitive symptoms (in children, only one symptom is necessary for a diagnosis of GAD): edginess or restlessness, tiring easily, more fatigued than usual, impaired concentration or feeling as though the mind goes blank, irritability (which may or may not be observable to others), increased muscle aches or soreness, difficulty sleeping (due to trouble falling asleep or staying asleep, restlessness at night, or unsatisfying sleep).

In some embodiments, the composition of the invention is for use in the treatment or one or more of the above symptoms of GAD. Treatment of one or more symptoms of GAD can be determined by clinically assessing a subject pre and post-administration of the composition to ascertain an improvement in one or more symptoms associated with GAD. In some embodiments, the composition of the invention improves the symptoms associated with GAD according to a symptomatic or diagnostic test and/or scale. In certain embodiments, the test or scale is that specified in the DSM-5. In certain embodiments, the scale is the Hamilton Anxiety Rating Scale (HAM-A).

In some embodiments, the compositions of the invention improve the Clinical Global Impression-Global Improvement (CGI-I) scale for assessing psychiatric and neurological disorders. In some embodiments, the compositions of the invention display a positive effect on global social and occupational impairment of the subject clinically diagnosed with GAD.

In some embodiments, the composition of the invention is for simultaneous, sequential or separate use with a selective serotonin reuptake inhibitor (SSRI) in the treatment or prevention of anxiety.

In some embodiments, the invention relates to an SSRI for simultaneous, sequential or separate use with the composition of the invention in the treatment or prevention of anxiety.

In some embodiments, the invention relates to an SSRI for use in the treatment or prevention of anxiety in a subject, wherein the subject is to be administered or has been administered with the composition of the invention.

In some embodiments, the composition of the invention is for use in the in the treatment or prevention of anxiety, wherein the subject is to be administered or has been administered with an SSRI.

Exemplary SSRIs suitable for use in the invention include, but are not limited to citalopram, dapoxetine, escitalopram, fluoxetine, fluvoxamine, paroxetine, sertraline and vortioxetine.

In some embodiments, the composition of the invention is for use in the treatment or prevention of anxiety in combination with one or more therapies selected from the list consisting of: cognitive behavioural therapy, applied relaxation, a tricyclic antidepressant, a serotonin-noradrenaline reuptake inhibitor, a monoamine oxidase inhibitor, a benzodiazepine, or pregabalin.

Post-Traumatic Stress Disorder

The composition of the invention may be particularly useful in the treatment or prevention of Post-traumatic stress disorder (PTSD). PTSD is a specific type of anxiety disorder. Treatment of PTSD using serotonin reuptake inhibitors has been shown to be therapeutically effective [27]. Therefore, the composition of the invention may be effective in the treatment of PTSD.

In some embodiments, the composition of the invention is for use in the treatment or prevention of PTSD in a subject, wherein, when administered to the subject the composition increases the expression of tryptophan hydroxylase. In some embodiments, when administered to the subject, the composition increases the expression of tryptophan hydroxylase 1 in the subject. In some embodiments, when administered to the subject, the composition increases the expression of tryptophan hydroxylase 2 in the subject. In some embodiments, when administered to the subject, the composition increases the expression of tryptophan hydroxylase 1 and 2 in the subject. In some embodiments, the composition increases the expression of tryptophan hydroxylase expression in neurons. In some embodiments, the composition increases the expression of tryptophan hydroxylase expression in serotonergic neurons. In some embodiments, the composition of the invention increases the expression of tryptophan hydroxylase in serotonergic neurons in the raphe nuclei of the subject. The raphe nuclei comprise a moderate-size cluster of nuclei found in the brain stem. One of the main functions of these nuclei is to release serotonin to the rest of the brain. Therefore, increasing the expression of tryptophan hydroxylase in neurons of the raphe nuclei is beneficial in increasing the production of serotonin accessible to the brain.

In some embodiments, the composition of the invention is for use in the treatment or prevention of PTSD in a subject, wherein, when administered to the subject the composition increases the expression of serotonin transporter in the subject. In some embodiments, the composition increases the expression of serotonin transporter in neurons in the subject. In some embodiments, the composition increases the expression of serotonin transporter in serotonergic neurons in the subject. In some embodiments, the composition of the invention increases the expression of serotonin transporter in serotonergic neurons in the raphe nuclei of the subject.

In some embodiments, the composition of the invention is for use in the treatment or prevention of PTSD in a subject, wherein the subject has been diagnosed with PTSD. According to the DSM-IV-TR, a PTSD diagnosis can be made if: (1) the patient experienced, witnessed, or was confronted with an event or events that involved actual or threatened death or serious injury, or a threat to the physical integrity of self or others and the response involved intense fear, helplessness, or horror; (2) as a consequence of the traumatic event, the patient experiences at least one re-experiencing/intrusion symptom, three avoidance/numbing symptoms, and two hyper-arousal symptoms, and the duration of the symptoms is for more than one month; and (3) the symptoms cause clinically significant distress or impairment in social, occupational, or other important areas of functioning.

In some embodiments, the composition of the invention is for use in the treatment or one or more symptoms of PTSD consisting of: recurrent and intrusive trauma recollections, recurrent and distressing dreams of the traumatic event, acting or feeling as if the traumatic event were recurring, distress when exposed to trauma reminders, physiological reactivity when exposed to trauma reminders, markedly diminished interest in significant activities, feelings of detachment or estrangement from others, restricted range of affect, sense of a foreshortened future, social anxiety, anxiety with unfamiliar surroundings, difficulty falling or staying asleep, irritability or outbursts of anger, difficulty concentrating, hyper-vigilance, problems with pain perception, pain tolerance, and exaggerated startle response.

Treatment of one or more symptoms of PTSD can be determined by clinically assessing a subject pre and post-administration of the composition to ascertain an improvement in one or more symptoms associated with PTSD. In some embodiments, the composition of the invention improves the symptoms associated with PTSD according to a symptomatic or diagnostic test and/or scale. In certain embodiments, the test or scale is the Clinician-Administered PTSD Scale and or the Clinician-Administered PTSD Scale Part 2. In certain embodiments, the test or scale is that specified in the DSM-IV-TR.

In some embodiments, the compositions of the invention improve the Clinical Global Impression-Global Improvement (CGI-I) scale for assessing psychiatric and neurological disorders. In some embodiments, the compositions of the invention display a positive effect on global social and occupational impairment of the subject clinically diagnosed with PTSD.

In some embodiments, the composition of the invention is for simultaneous, sequential or separate use with an SSRI in the treatment of PTSD.

In some embodiments, the invention relates to an SSRI for simultaneous, sequential or separate use with the composition of the invention in the treatment of PTSD.

In some embodiments, the invention relates to an SSRI for use in the treatment or prevention of PTSD in a subject, wherein the subject is to be administered or has been administered with the composition of the invention.

In some embodiments, the composition of the invention is for use in the treatment of PTSD in a subject, wherein the subject is to be administered or has been administered with an SSRI.

Exemplary SSRIs suitable for use in the invention include, but are not limited to citalopram, dapoxetine, escitalopram, fluoxetine, fluvoxamine, paroxetine, sertraline and vortioxetine.

In some embodiments, the composition of the invention is for use in the treatment of PTSD in combination with one or more therapies selected from the list consisting of: trauma-focussed cognitive behavioural therapy, eye movement densitisation and reprocessing, trycyclic antidepressants, serotonin-noradrenaline reuptake inhibitors, noradrenaline and specific serotonergic antidepressants and monoamine oxidase inhibitors.

Obsessive Compulsive Disorder

The composition of the invention may be particularly useful in the treatment or prevention of obsessive compulsive disorder (OCD). OCD is a specific type of psychiatric disorder where subjects feel the need to check and perform things repeatedly (i.e. perform “rituals”). Serotonin deficit has been implicated in the pathology of OCD [28]. Therefore, the composition of the invention may be effective in the treatment of OCD, by increasing the bioavailability of serotonin.

In some embodiments, the composition of the invention is for use in the treatment or prevention of OCD in a subject, wherein, when administered to the subject the composition increases the expression of tryptophan hydroxylase. In some embodiments, when administered to the subject, the composition increases the expression of tryptophan hydroxylase 1 in the subject. In some embodiments, when administered to the subject, the composition increases the expression of tryptophan hydroxylase 2 in the subject. In some embodiments, when administered to the subject, the composition increases the expression of tryptophan hydroxylase 1 and 2 in the subject. In some embodiments, the composition increases the expression of tryptophan hydroxylase expression in neurons. In some embodiments, the composition increases the expression of tryptophan hydroxylase expression in serotonergic neurons. In some embodiments, the composition of the invention increases the expression of tryptophan hydroxylase in serotonergic neurons in the raphe nuclei of the subject. The raphe nuclei comprise a moderate-size cluster of nuclei found in the brain stem. One of the main functions of these nuclei is to release serotonin to the rest of the brain. Therefore, increasing the expression of tryptophan hydroxylase in neurons of the raphe nuclei is beneficial in increasing the production of serotonin accessible to the brain. In some embodiments, when administered to a subject, the composition increases the production of serotonin in the subject via the tryptophan hydroxylase pathway.

In some embodiments, the composition of the invention is for use in the treatment or prevention of OCD in a subject, wherein, when administered to the subject the composition increases the expression of serotonin transporter in the subject. In some embodiments, the composition increases the expression of serotonin transporter in neurons in the subject. In some embodiments, the composition increases the expression of serotonin transporter in serotonergic neurons in the subject. In some embodiments, the composition of the invention increases the expression of serotonin transporter in serotonergic neurons in the raphe nuclei of the subject.

In some embodiments, the composition of the invention is for use in the treatment or prevention of OCD in a subject, wherein the subject has been diagnosed with OCD. According to the DSM-5, OCD diagnosis can be made if. (1) the subject has obsessions and compulsions; (2) the obsessions and compulsions significantly impact the daily life of the subject; and (3) the subject may not realize that the obsessions and compulsions are excessive or unreasonable. The obsessions must be intrusive, repetitive and persistent thoughts, urges, or images that cause distress; do not just excessively focus on real problems in your life; cannot be successfully suppressed or ignored by the subject; and (iv) that cannot be reconciled that the thoughts, urges or images do not pose a true threat to the subject. The compulsions must be excessive and repetitive ritualistic behaviour that the subject feels they must perform, or something bad will happen; take up at least one hour per day; and must be performed to reduce severe anxiety caused by obsessive thoughts.

In some embodiments, the composition of the invention is for use in the treatment or one or more obsessions and/or compulsions of the OCD

Treatment of one or more obsessions and/or compulsions can be determined by clinically assessing a subject pre and post-administration of the composition to ascertain an improvement in one or more obsessions and/or compulsions. In some embodiments, the composition of the invention improves the obsessions and/or compulsions according to a symptomatic or diagnostic test and/or scale. In certain embodiments, the test or scale specified in the DSM-V.

In some embodiments, the compositions of the invention improve the Clinical Global Impression-Global Improvement (CGI-I) scale for assessing psychiatric and neurological disorders. In some embodiments, the compositions of the invention display a positive effect on global social and occupational impairment of the subject clinically diagnosed with OCD.

In some embodiments, the composition of the invention is for simultaneous, sequential or separate use with an SSRI in the treatment of OCD.

In some embodiments, the invention relates to an SSRI for simultaneous, sequential or separate use with the composition of the invention in the treatment of OCD.

In some embodiments, the invention relates to an SSRI for use in the treatment or prevention of OCD in a subject, wherein the subject is to be administered or has been administered with the composition of the invention.

In some embodiments, the composition of the invention is for use in the treatment of OCD in a subject, wherein the subject is to be administered or has been administered with an SSRI.

Exemplary SSRIs suitable for use in the invention include, but are not limited to citalopram, dapoxetine, escitalopram, fluoxetine, fluvoxamine, paroxetine, sertraline and vortioxetine.

In some embodiments, the composition of the invention is for use in the treatment of OCD in combination with one or more therapies selected from the list consisting of: exposure and response prevention.

Modes of Administration

Preferably, the compositions of the invention are to be administered to the gastrointestinal tract in order to enable delivery to and/or partial or total colonisation of the intestine with the bacterial strain of the invention. Generally, the compositions of the invention are administered orally, but they may be administered rectally, intranasally, or via buccal or sublingual routes.

In certain embodiments, the compositions of the invention may be administered as a foam, as a spray or a gel.

In certain embodiments, the compositions of the invention may be administered as a suppository, such as a rectal suppository, for example in the form of a theobroma oil (cocoa butter), synthetic hard fat (e.g. suppocire, witepsol), glycero-gelatin, polyethylene glycol, or soap glycerin composition.

In certain embodiments, the composition of the invention is administered to the gastrointestinal tract via a tube, such as a nasogastric tube, orogastric tube, gastric tube, jejunostomy tube (J tube), percutaneous endoscopic gastrostomy (PEG), or a port, such as a chest wall port that provides access to the stomach, jejunum and other suitable access ports.

The compositions of the invention may be administered once, or they may be administered sequentially as part of a treatment regimen. In certain embodiments, the compositions of the invention are to be administered daily.

In certain embodiments of the invention, treatment according to the invention is accompanied by assessment of the patient's gut microbiota. Treatment may be repeated if delivery of and/or partial or total colonisation with the strain of the invention is not achieved such that efficacy is not observed, or treatment may be ceased if delivery and/or partial or total colonisation is successful and efficacy is observed.

In certain embodiments, the composition of the invention may be administered to a pregnant animal, for example a mammal such as a human in order to prevent a disorder associated with serotonin deficiency developing in her child in utero and/or after it is born.

The compositions of the invention may be administered to a patient that has been diagnosed with a disorder characterised by serotonin deficiency, or that has been identified as being at risk of a disorder characterised by serotonin deficiency. The compositions may also be administered as a prophylactic measure to prevent the development of disorder characterised by serotonin deficiency in a healthy patient.

The compositions of the invention may be administered to a patient that has been identified as having an abnormal gut microbiota. For example, the patient may have reduced or absent colonisation by Megasphaera, and in particular Megasphaera massiliensis.

The compositions of the invention may be administered as a food product, such as a nutritional supplement.

Generally, the compositions of the invention are for the treatment of humans, although they may be used to treat animals including monogastric mammals such as poultry, pigs, cats, dogs, horses or rabbits. The compositions of the invention may be useful for enhancing the growth and performance of animals. If administered to animals, oral gavage may be used.

Compositions

Generally, the composition of the invention comprises bacteria. In preferred embodiments of the invention, the composition is formulated in freeze-dried form. For example, the composition of the invention may comprise granules or gelatin capsules, for example hard gelatin capsules, comprising a bacterial strain of the invention.

Preferably, the composition of the invention comprises lyophilised bacteria. Lyophilisation of bacteria is a well-established procedure and relevant guidance is available in, for example, references [29], [ ], [31]].

Alternatively, the composition of the invention may comprise a live, active bacterial culture.

In some embodiments, the bacterial strain in the composition of the invention has not been inactivated, for example, has not been heat-inactivated. In some embodiments, the bacterial strain in the composition of the invention has not been killed, for example, has not been heat-killed. In some embodiments, the bacterial strain in the composition of the invention has not been attenuated, for example, has not been heat-attenuated. For example, in some embodiments, the bacterial strain in the composition of the invention has not been killed, inactivated and/or attenuated. For example, in some embodiments, the bacterial strain in the composition of the invention is live. For example, in some embodiments, the bacterial strain in the composition of the invention is viable. For example, in some embodiments, the bacterial strain in the composition of the invention is capable of partially or totally colonising the intestine. For example, in some embodiments, the bacterial strain in the composition of the invention is viable and capable of partially or totally colonising the intestine.

In some embodiments, the composition comprises a mixture of live bacterial strains and bacterial strains that have been killed.

In preferred embodiments, the composition of the invention is encapsulated to enable delivery of the bacterial strain to the intestine. Encapsulation protects the composition from degradation until delivery at the target location through, for example, rupturing with chemical or physical stimuli such as pressure, enzymatic activity, or physical disintegration, which may be triggered by changes in pH. Any appropriate encapsulation method may be used. Exemplary encapsulation techniques include entrapment within a porous matrix, attachment or adsorption on solid carrier surfaces, self-aggregation by flocculation or with cross-linking agents, and mechanical containment behind a microporous membrane or a microcapsule. Guidance on encapsulation that may be useful for preparing compositions of the invention is available in, for example, references [32] and [33].

The composition may be administered orally and may be in the form of a tablet, capsule or powder. Encapsulated products are preferred because Megasphaera are anaerobes. Other ingredients (such as vitamin C, for example), may be included as oxygen scavengers and prebiotic substrates to improve the delivery and/or partial or total colonisation and survival in vivo. Alternatively, the probiotic composition of the invention may be administered orally as a food or nutritional product, such as milk or whey based fermented dairy product, or as a pharmaceutical product.

The composition may be formulated as a probiotic.

A composition of the invention includes a therapeutically effective amount of a bacterial strain of the invention. A therapeutically effective amount of a bacterial strain is sufficient to exert a beneficial effect upon a patient. A therapeutically effective amount of a bacterial strain may be sufficient to result in delivery to and/or partial or total colonisation of the patient's intestine.

A suitable daily dose of the bacteria, for example for an adult human, may be from about 1×10³ to about 1×10¹¹ colony forming units (CFU); for example, from about 1×10⁷ to about 1×10¹⁰ CFU; in another example from about 1×10⁶ to about 1×10¹⁰ CFU.; in another example from about 1×10⁷ to about 1×10¹¹ CFU; in another example from about 1×10′ to about 1×10¹⁰ CFU; in another example from about 1×10′ to about 1×10¹¹ CFU.

In certain embodiments, the dose of the bacteria is at least 10⁹ cells per day, such as at least 10¹⁰, at least 10¹¹, or at least 10¹² cells per day.

In certain embodiments, the composition contains the bacterial strain in an amount of from about 1×10⁶ to about 1×10¹¹ CFU/g, respect to the weight of the composition; for example, from about 1×10′ to about 1×10¹⁰ CFU/g. The dose may be, for example, 1 g, 3 g, 5 g, and 10 g.

In certain embodiments, the invention provides the above pharmaceutical composition, wherein the amount of the bacterial strain is from about 1×10 to about 1×10¹ colony forming units per gram with respect to a weight of the composition.

Typically, a probiotic, such as the composition of the invention, is optionally combined with at least one suitable prebiotic compound. A prebiotic compound is usually a non-digestible carbohydrate such as an oligo- or polysaccharide, or a sugar alcohol, which is not degraded or absorbed in the upper digestive tract. Known prebiotics include commercial products such as inulin and transgalacto-oligosaccharides.

In certain embodiments, the probiotic composition of the present invention includes a prebiotic compound in an amount of from about 1 to about 30% by weight, respect to the total weight composition, (e.g. from 5 to 20% by weight). Carbohydrates may be selected from the group consisting of: fructo-oligosaccharides (or FOS), short-chain fructo-oligosaccharides, inulin, isomalt-oligosaccharides, pectins, xylo-oligosaccharides (or XOS), chitosan-oligosaccharides (or COS), beta-glucans, arable gum modified and resistant starches, polydextrose, D-tagatose, acacia fibers, carob, oats, and citrus fibers. In one aspect, the prebiotics are the short-chain fructo-oligosaccharides (for simplicity shown herein below as FOSs-c.c); said FOSs-c.c. are not digestible carbohydrates, generally obtained by the conversion of the beet sugar and including a saccharose molecule to which three glucose molecules are bonded.

In certain embodiments, the compositions of the invention are used in combination with another therapeutic compound for treating or preventing the disorder characterised by serotonin deficiency. In some embodiments, the compositions of the invention are administered with nutritional supplements that modulate neuroprotection or neuroproliferation. In preferred embodiments, the nutritional supplements comprise or consist of nutritional vitamins. In certain embodiments, the vitamins are vitamin B6, magnesium, dimethylglycine (vitamin B16) and vitamin C. In certain embodiments, the compositions of the invention are administered in combination with another probiotic.

In certain embodiments, the compositions of the invention are for use in enhancing the effect of a second agent on a disorder characterised by serotonin deficiency.

The compositions of the invention may comprise pharmaceutically acceptable excipients or carriers. Examples of such suitable excipients may be found in the reference [34]. Acceptable carriers or diluents for therapeutic use are well known in the pharmaceutical art and are described, for example, in reference [35]. Examples of suitable carriers include lactose, starch, glucose, methyl cellulose, magnesium stearate, mannitol, sorbitol and the like. Examples of suitable diluents include ethanol, glycerol and water. The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s). Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol. Examples of suitable lubricants include sodium oleate, sodium stearate, magnesium stearate, sodium benzoate, sodium acetate, sodium chloride and the like. Preservatives, stabilizers, dyes and even flavouring agents may be provided in the pharmaceutical composition. Examples of preservatives include sodium benzoate, sorbic acid and esters of p-hydroxybenzoic acid. Antioxidants and suspending agents may be also used.

The compositions of the invention may be formulated as a food product. For example, a food product may provide nutritional benefit in addition to the therapeutic effect of the invention, such as in a nutritional supplement. Similarly, a food product may be formulated to enhance the taste of the composition of the invention or to make the composition more attractive to consume by being more similar to a common food item, rather than to a pharmaceutical composition. In certain embodiments, the composition of the invention is formulated as a milk-based product. The term “milk-based product” means any liquid or semi-solid milk- or whey-based product having a varying fat content. The milk-based product can be, e.g., cow's milk, goat's milk, sheep's milk, skimmed milk, whole milk, milk recombined from powdered milk and whey without any processing, or a processed product, such as yoghurt, curdled milk, curd, sour milk, sour whole milk, butter milk and other sour milk products. Another important group includes milk beverages, such as whey beverages, fermented milks, condensed milks, infant or baby milks; flavoured milks, ice cream; milk-containing food such as sweets.

In some embodiments, the compositions of the invention comprise one or more bacterial strains of the genus Megasphaera and do not contain bacteria from any other genera, or which comprise only de minimis or biologically irrelevant amounts of bacteria from another genera. Thus, in some embodiments, the invention provides a composition comprising one or more bacterial strains of the genus Megasphaera, which does not contain bacteria from any other genera or which comprises only de minimis or biologically irrelevant amounts of bacteria from another genera, for use in therapy.

In some embodiments, the compositions of the invention comprise one or more bacterial strains of the species Megasphaera massiliensis and do not contain bacteria from any other species, or which comprise only de minimis or biologically irrelevant amounts of bacteria from another species. Thus, in some embodiments, the invention provides a composition comprising one or more bacterial strains of the species Megasphaera massiliensis, which does not contain bacteria from any other species or which comprises only de minimis or biologically irrelevant amounts of bacteria from another species, for use in therapy.

In some embodiments, the compositions of the invention comprise one or more bacterial strains of the species Megasphaera massiliensis and do not contain bacteria from any other Megasphaera species, or which comprise only de minimis or biologically irrelevant amounts of bacteria from another Megasphaera species. Thus, in some embodiments, the invention provides a composition comprising one or more bacterial strains of the species Megasphaera massiliensis, which does not contain bacteria from any other Megasphaera species or which comprises only de minimis or biologically irrelevant amounts of bacteria from another Megasphaera species, for use in therapy.

In certain embodiments, the compositions of the invention contain a single bacterial strain or species and do not contain any other bacterial strains or species. Such compositions may comprise only de minimis or biologically irrelevant amounts of other bacterial strains or species. Such compositions may be a culture that is substantially free from other species of organism.

In some embodiments, the invention provides a composition comprising a single bacterial strain of the genus Megasphaera, which does not contain bacteria from any other strains or which comprises only de minimis or biologically irrelevant amounts of bacteria from another strain for use in therapy.

In some embodiments, the invention provides a composition comprising a single bacterial strain of the species Megasphaera massiliensis, which does not contain bacteria from any other strains or which comprises only de minimis or biologically irrelevant amounts of bacteria from another strain for use in therapy.

In some embodiments, the compositions of the invention comprise more than one bacterial strain. For example, in some embodiments, the compositions of the invention comprise more than one strain from within the same species (e.g. more than 1, 2, 3, 4, 5, 6, 7, 8, 9, 10, 15, 20, 25, 30, 35, 40 or 45 strains), and, optionally, do not contain bacteria from any other species. In some embodiments, the compositions of the invention comprise less than 50 strains from within the same species (e.g. less than 45, 40, 35, 30, 25, 20, 15, 12, 10, 9, 8, 7, 6, 5, 4 or 3 strains), and, optionally, do not contain bacteria from any other species. In some embodiments, the compositions of the invention comprise 1-40, 1-30, 1-20, 1-19, 1-18, 1-15, 1-10, 1-9, 1-8, 1-7, 1-6, 1-5, 1-4, 1-3, 1-2, 2-50, 2-40, 2-30, 2-20, 2-15, 2-10, 2-5, 6-30, 6-15, 16-25, or 31-50 strains from within the same species and, optionally, do not contain bacteria from any other species. The invention comprises any combination of the foregoing.

In some embodiments, the composition comprises a microbial consortium. For example, in some embodiments, the composition comprises the Megasphaera bacterial strain as part of a microbial consortium. For example, in some embodiments, the Megasphaera bacterial strain is present in combination with one or more (e.g. at least 2, 3, 4, 5, 10, 15 or 20) other bacterial strains from other genera with which it can live symbiotically in vivo in the intestine. For example, in some embodiments, the composition comprises a bacterial strain of Megasphaera in combination with a bacterial strain from a different genus. In some embodiments, the microbial consortium comprises two or more bacterial strains obtained from a faeces sample of a single organism, e.g. a human. In some embodiments, the microbial consortium is not found together in nature. For example, in some embodiments, the microbial consortium comprises bacterial strains obtained from faeces samples of at least two different organisms. In some embodiments, the two different organisms are from the same species, e.g. two different humans. In some embodiments, the two different organisms are an infant human and an adult human. In some embodiments, the two different organisms are a human and a non-human mammal.

In alternative embodiments, compositions of the invention comprise 16, 15, 14, 13, 12, 11, 10, 9, 8, 7, 6, 5 or fewer distinct bacterial species. In certain embodiments, the composition comprises 4 or fewer distinct bacterial species. In certain embodiments, the composition comprises 3 or fewer distinct bacterial species. In certain embodiments, the composition comprises 2 or fewer distinct bacterial species. In certain embodiments, the composition comprises a species of Megasphaera, in particular Megasphaera massiliensis, and no other bacterial species. In preferred embodiments, the compositions of the invention comprise a single strain Megasphaera, in particular a single strain of Megasphaera massiliensis, and no other bacterial strains or species. Such compositions may comprise only de minimis or biologically irrelevant amounts of other bacterial strains or species. Strikingly, the examples demonstrate that compositions comprising only a single strain of the invention can have potent effects (see e.g. Examples 1 and 3), with no reliance on other strains or species.

In some embodiments, the composition of the invention additionally comprises a bacterial strain that has the same safety and therapeutic efficacy characteristics as strain NCIMB 42787, but which is not NCIMB 42787, or which is not a Megasphaera massiliensis.

In some embodiments in which the composition of the invention comprises more than one bacterial strain, species or genus, the individual bacterial strains, species or genera may be for separate, simultaneous or sequential administration. For example, the composition may comprise all of the more than one bacterial strain, species or genera, or the bacterial strains, species or genera may be stored separately and be administered separately, simultaneously or sequentially. In some embodiments, the more than one bacterial strains, species or genera are stored separately but are mixed together prior to use.

In some embodiments, the bacterial strain for use in the invention is obtained from human adult faeces. In some embodiments in which the composition of the invention comprises more than one bacterial strain, all of the bacterial strains are obtained from human adult faeces or if other bacterial strains are present they are present only in de minimis amounts. The bacteria may have been cultured subsequent to being obtained from the human adult faeces and being used in a composition of the invention.

As mentioned above, in some embodiments, the one or more Megasphaera bacterial strains is/are the only therapeutically active agent(s) in a composition of the invention. In some embodiments, the bacterial strain(s) in the composition is/are the only therapeutically active agent(s) in a composition of the invention.

The compositions for use in accordance with the invention may or may not require marketing approval.

In certain embodiments, the invention provides the above pharmaceutical composition, wherein said bacterial strain is lyophilised. In certain embodiments, the invention provides the above pharmaceutical composition, wherein said bacterial strain is spray dried. In certain embodiments, the invention provides the above pharmaceutical composition, wherein the bacterial strain is lyophilised or spray dried and wherein it is live. In certain embodiments, the invention provides the above pharmaceutical composition, wherein the bacterial strain is lyophilised or spray dried and wherein it is viable. In certain embodiments, the invention provides the above pharmaceutical composition, wherein the bacterial strain is lyophilised or spray dried and wherein it is capable of partially or totally colonising the intestine. In certain embodiments, the invention provides the above pharmaceutical composition, wherein the bacterial strain is lyophilised or spray dried and wherein it is viable and capable of partially or totally colonising the intestine.

In some cases, the lyophilised bacterial strain is reconstituted prior to administration. In some cases, the reconstitution is by use of a diluent described herein.

The compositions of the invention can comprise pharmaceutically acceptable excipients, diluents or carriers.

In certain embodiments, the invention provides a pharmaceutical composition comprising: a bacterial strain of the invention; and a pharmaceutically acceptable excipient, carrier or diluent; wherein the bacterial strain is in an amount sufficient to treat a disorder characterised by serotonin deficiency when administered to a subject in need thereof.

In certain embodiments, the invention provides pharmaceutical composition comprising: a bacterial strain of the invention; and a pharmaceutically acceptable excipient, carrier or diluent; wherein the bacterial strain is in an amount sufficient to treat or prevent a disorder characterised by serotonin deficiency.

In certain embodiments, the invention provides the above pharmaceutical composition, wherein the amount of the bacterial strain is from about 1×10³ to about 1×10¹¹ colony forming units per gram with respect to a weight of the composition.

In certain embodiments, the invention provides the above pharmaceutical composition, wherein the composition is administered at a dose of 1 g, 3 g, 5 g or 10 g.

In certain embodiments, the invention provides the above pharmaceutical composition, wherein the composition is administered by a method selected from the group consisting of oral, rectal, subcutaneous, nasal, buccal, and sublingual.

In certain embodiments, the invention provides the above pharmaceutical composition, comprising a carrier selected from the group consisting of lactose, starch, glucose, methyl cellulose, magnesium stearate, mannitol and sorbitol.

In certain embodiments, the invention provides the above pharmaceutical composition, comprising a diluent selected from the group consisting of ethanol, glycerol and water.

In certain embodiments, the invention provides the above pharmaceutical composition, comprising an excipient selected from the group consisting of starch, gelatin, glucose, anhydrous lactose, free-flow lactose, beta-lactose, corn sweetener, acacia, tragacanth, sodium alginate, carboxymethyl cellulose, polyethylene glycol, sodium oleate, sodium stearate, magnesium stearate, sodium benzoate, sodium acetate and sodium chloride.

In certain embodiments, the invention provides the above pharmaceutical composition, further comprising at least one of a preservative, an antioxidant and a stabilizer.

In certain embodiments, the invention provides the above pharmaceutical composition, comprising a preservative selected from the group consisting of sodium benzoate, sorbic acid and esters of p-hydroxybenzoic acid.

In certain embodiments, the invention provides the above pharmaceutical composition, wherein said bacterial strain is lyophilised.

In certain embodiments, the invention provides the above pharmaceutical composition, wherein when the composition is stored in a sealed container at about 4° C. or about 25° C. and the container is placed in an atmosphere having 50% relative humidity, at least 80% of the bacterial strain as measured in colony forming units, remains after a period of at least about: 1 month, 3 months, 6 months, 1 year, 1.5 years, 2 years, 2.5 years or 3 years.

In some embodiments, the composition of the invention is provided in a sealed container comprising a composition as described herein. In some embodiments, the sealed container is a sachet or bottle. In some embodiments, the composition of the invention is provided in a syringe comprising a composition as described herein.

The composition of the present invention may, in some embodiments, be provided as a pharmaceutical formulation. For example, the composition may be provided as a tablet or capsule. In some embodiments, the capsule is a gelatine capsule (“gel-cap”).

In some embodiments, the compositions of the invention are administered orally. Oral administration may involve swallowing, so that the compound enters the gastrointestinal tract, and/or buccal, lingual, or sublingual administration by which the compound enters the blood stream directly from the mouth.

Pharmaceutical formulations suitable for oral administration include solid plugs, solid microparticulates, semi-solid and liquid (including multiple phases or dispersed systems) such as tablets; soft or hard capsules containing multi- or nano-particulates, liquids (e.g. aqueous solutions), emulsions or powders; lozenges (including liquid-filled); chews; gels; fast dispersing dosage forms; films; ovules; sprays; and buccal/mucoadhesive patches.

In some embodiments the pharmaceutical formulation is an enteric formulation, i.e. a gastro-resistant formulation (for example, resistant to gastric pH) that is suitable for delivery of the composition of the invention to the intestine by oral administration. Enteric formulations may be particularly useful when the bacteria or another component of the composition is acid-sensitive, e.g. prone to degradation under gastric conditions.

In some embodiments, the enteric formulation comprises an enteric coating. In some embodiments, the formulation is an enteric-coated dosage form. For example, the formulation may be an enteric-coated tablet or an enteric-coated capsule, or the like. The enteric coating may be a conventional enteric coating, for example, a conventional coating for a tablet, capsule, or the like for oral delivery. The formulation may comprise a film coating, for example, a thin film layer of an enteric polymer, e.g. an acid-insoluble polymer.

In some embodiments, the enteric formulation is intrinsically enteric, for example, gastro-resistant without the need for an enteric coating. Thus, in some embodiments, the formulation is an enteric formulation that does not comprise an enteric coating. In some embodiments, the formulation is a capsule made from a thermogelling material. In some embodiments, the thermogelling material is a cellulosic material, such as methylcellulose, hydroxymethylcellulose or hydroxypropylmethylcellulose (HPMC). In some embodiments, the capsule comprises a shell that does not contain any film forming polymer. In some embodiments, the capsule comprises a shell and the shell comprises hydroxypropylmethylcellulose and does not comprise any film forming polymer (e.g. see [36]). In some embodiments, the formulation is an intrinsically enteric capsule (for example, Vcaps® from Capsugel).

In some embodiments, the formulation is a soft capsule. Soft capsules are capsules which may, owing to additions of softeners, such as, for example, glycerol, sorbitol, maltitol and polyethylene glycols, present in the capsule shell, have a certain elasticity and softness. Soft capsules can be produced, for example, on the basis of gelatine or starch. Gelatine-based soft capsules are commercially available from various suppliers. Depending on the method of administration, such as, for example, orally or rectally, soft capsules can have various shapes, they can be, for example, round, oval, oblong or torpedo-shaped. Soft capsules can be produced by conventional processes, such as, for example, by the Scherer process, the Accogel process or the droplet or blowing process.

Culturing Methods

The bacterial strains for use in the present invention can be cultured using standard microbiology techniques as detailed in, for example, references [37], [ ] and [39].

The solid or liquid medium used for culture may be YCFA+ agar or YCFA medium. YCFA medium may include (per 100 ml, approximate values): Casitone (1.0 g), yeast extract (0.25 g), NaHCO₃ (0.4 g), cysteine (0.1 g), K₂HPO₄ (0.045 g), KH₂PO₄ (0.045 g), NaCl (0.09 g), (NH₄)₂SO₄ (0.09 g), MgSO₄ 7H₂O (0.009 g), CaCl₂ (0.009 g), resazurin (0.1 mg), hemin (1 mg), biotin (1 μg), cobalamin (1 μg), p-aminobenzoic acid (3 μg), folic acid (5 μg), and pyridoxamine (15 μg).

Bacterial Strains for Use in Vaccine Compositions

The inventors have identified that the bacterial strains of the invention are useful for treating or preventing disorders characterised by serotonin deficiency. This is likely to be a result of the effect that the bacterial strains of the invention have on the host immune system. Therefore, the compositions of the invention may also be useful for preventing disorders characterised by serotonin deficiency, when administered as vaccine compositions. In certain such embodiments, the bacterial strains of the invention may be killed, inactivated or attenuated. In certain such embodiments, the compositions may comprise a vaccine adjuvant. In certain embodiments, the compositions are for administration via injection, such as via subcutaneous injection.

General

The practice of the present invention will employ, unless otherwise indicated, conventional methods of chemistry, biochemistry, molecular biology, immunology and pharmacology, within the skill of the art. Such techniques are explained fully in the literature. See, e.g., references [40] and [41,47], etc.

The term “comprising” encompasses “including” as well as “consisting” e.g. a composition “comprising” X may consist exclusively of X or may include something additional e.g. X+Y.

The term “about” in relation to a numerical value x is optional and means, for example, x+10%.

The word “substantially” does not exclude “completely” e.g. a composition which is “substantially free” from Y may be completely free from Y. Where necessary, the word “substantially” may be omitted from the definition of the invention.

References to a percentage sequence identity between two nucleotide sequences means that, when aligned, that percentage of nucleotides are the same in comparing the two sequences. This alignment and the percent homology or sequence identity can be determined using software programs known in the art, for example those described in section 7.7.18 of ref. [48]. A preferred alignment is determined by the Smith-Waterman homology search algorithm using an affine gap search with a gap open penalty of 12 and a gap extension penalty of 2, BLOSUM matrix of 62. The Smith-Waterman homology search algorithm is disclosed in ref. [49].

Unless specifically stated, a process or method comprising numerous steps may comprise additional steps at the beginning or end of the method, or may comprise additional intervening steps. Also, steps may be combined, omitted or performed in an alternative order, if appropriate.

Various embodiments of the invention are described herein. It will be appreciated that the features specified in each embodiment may be combined with other specified features, to provide further embodiments. In particular, embodiments highlighted herein as being suitable, typical or preferred may be combined with each other (except when they are mutually exclusive).

### MODES FOR CARRYING OUT THE INVENTION

### Example 1—Neurochemical Production—Metabolites in the Brain

Background

The level of neurochemical factors, neuropeptides and neurotransmitters that play a key role in neurological processes were measured during the ex vivo screening in brain tissue of mice fed with Megasphaera massiliensis strain NCIMB 42787. Imbalance of these chemicals, for example, a deficiency (for example, sub-optimal levels) of serotonin has been implicated in the pathology of numerous psychiatric disorders.

Methods

Animals

BALBc (Envigo, UK) adult male mice were group housed under a 12 h light-dark cycle; standard rodent chow and water were available ad libitum. All experiments were performed in accordance with European guidelines following approval by University College Cork Animal Ethics Experimentation Committee. Animals were 8 weeks old at the start of the experiment.

Study Design

Animals were allowed to habituate to their holding room for one week after arrival into the animal unit. They receive oral gavage (200 μL dose) of live biotherapeutics at a dose of 1×10′ CFU for 6 consecutive days between 15:00 and 17:00. On day 7, the animals were decapitated, and tissues are harvested for experimentation.

Tissue Collection

Animals were sacrificed in a random fashion regarding treatment and testing condition; sampling occurred between 9.00 a.m. and 1:00 p.m. Trunk blood was collected in potassium EDTA (Ethylene Diamine Tetra Acetic Acid) tubes and spun for 15 min at 4000 g. Plasma was isolated and stored at −80° C. for further analysis. The brain was quickly excised, dissected and each brain region was snap-frozen on dry ice and stored at −80° C. for further analysis.

Analysis

Neurochemical factor, neuropeptide and neurotransmitter concentrations were analysed by HPLC on samples from the brainstem. Briefly, brainstem tissue was sonicated in 500 μg of chilled mobile phase spiked with 4 ng/40 μg of N-Methyl 5-HT (Sigma Chemical Co., UK) as internal standard. The mobile phase contained 0.1 M citric acid, 5.6 mM octane-1-sulphonic acid (Sigma), 0.1 M sodium dihydrogen phosphate, 0.01 mM EDTA (Alkem/Reagecon, Cork) and 9% (v/v) methanol (Alkem/Reagecon) and was adjusted to pH 2.8 using 4 N sodium hydroxide (Alkem/Reagecon). Homogenates were then centrifuged for 15 min at 22,000×g at 4° C. and 40 μl of the supernatant injected onto the HPLC system which consisted of a SCL 10-Avp system controller, LECD 6A electrochemical detector (Shimadzu), a LC-10AS pump, a CTO-10A oven, a SIL-10A autoinjector (with sample cooler maintained at 40 C) and an online Gastorr Degasser (ISS, UK). A reverse-phase column (Kinetex 2.6 u C18 100×4.6 mm, Phenomenex) maintained at 30° C. was employed in the separation (Flow rate 0.9 ml/min). The glassy carbon working electrode combined with an Ag/AgCl reference electrode (Shimdazu) operated a +0.8 V and the chromatograms generated were analyzed using Class-VP 5 software (Shimadzu). The neurotransmitters were identified by their characteristic retention times as determined by standard injections, which run at regular intervals during the sample analysis. The ratios of peak heights of analyte versus internal standard were measured and compared with standard injection. Results were expressed as ng of neurotransmitter per g fresh weight of tissue.

Results—Neurotransmitter Production

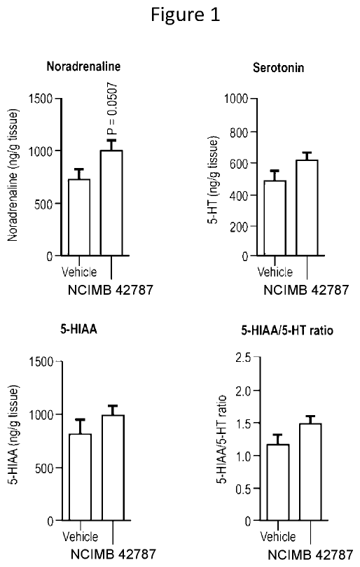

The results are shown in FIG. 1, which shows that in brains of mice fed with NCIMB 42787, noradrenaline (p=0.0507), serotonin and 5-HIAA levels were increased. Compositions of the invention therefore may be useful for treating or preventing disorders characterised by serotonin deficiency.

### Example 2—Tryptophan Hydroxylase Expression

Background

Tryptophan hydroxylase is an enzyme involved in the production of serotonin. The inventors thus sought to investigate whether the Megasphaera massiliensis strain NCIMB 42787 can induce the upregulated expression of the tryptophan hydroxylase genes TPH1 and TPH2 in neuron-like cells. This may explain how NCIMB 42787 increases the level of serotonin in vivo.

Material and Methods

Neuroblastoma SH-SY5Y cells were grown in 50% MEM 50% nutrient mixture F-12 Ham media supplemented with 2 mM L-glutamine, 10% heat-inactivated FBS, 100 U/ml penicillin and 100 μg/ml streptomycin. Cells were plated in 10 cm dishes at a density of 2×10⁶ cells. After 24 h rest, cells were treated in growth medium (containing 1% FBS) with 10% NCIMB 42787 supernatant or YCFA⁺, for 24 h. Cells were next collected, and total RNA was isolated according to the RNeasy mini kit protocol (Qiagen). cDNA was made using the high capacity cDNA reverse transcription kit (Applied Biosystems). Primer sequences are shown in Table 1. Gene expression was measured by qPCR. B-actin was used as internal control. Fold-change was calculated according to the 2\hat{ }(−ΔΔct) method.

A second set of similar experiments were conducted, except cells were plated in a six-well dish at a density of 0.5×10⁶ cells/well. After 24 h rest, cells were treated in growth medium (containing 1% FBS) with 5% bacterial supernatants or YCFA⁺, for 72 h. Total RNA was analysed as described above.

TABLE 1
primer sequences for TPH1 and β-actin

| Gene | Forward | Reverse |
|---|---|---|
| TPH1 | AAAGAGCGTACAGG | GTCTCACATATTGAG |
| | TTTTTC | TGCAG |
| | (SEQ ID NO: 3) | (SEQ ID NO: 4) |
| TPH2 | CACTATTGTGACGC | AGCTCAGAACCATAC |
| | TGAATC | ATGAG |
| | (SEQ ID NO: 9) | (SEQ ID NO: 10) |
| β-actin | GATCAAGATCATTG | TTGTCAAGAAAGGGT |
| | CTCCTC | GTAAC |
| | (SEQ ID NO: 5) | (SEQ ID NO: 6) |

Controls where cells were either left untreated or incubated for an equivalent time in YCFA⁺ medium were performed alongside. YCFA⁺ medium has the following composition:

| | |
|---|---|
| Bacto casitione | 1.0 g |
| Yeast extract | 0.25 g |
| Sodium hydrogen carbonate | 0.4 g |
| Glucose | 0.2 g |
| Cellobiose | 0.2 g |
| Soluble starch | 0.2 g |
| Mineral solution 1 | 15 ml |
| Mineral solution 2 | 15 ml |
| SCFA solution | 0.31 ml |
| Haemin solution | 1 ml |
| Vitamin solution 1 | 100 μl |
| Vitamin solution 2 | 100 μl |
| Resazurin solution | 0.1 ml |
| Cysteine | 0.1 g |
| d. H2O to a total volume of: | 100 m |

Mineral solution 1: K₂HPO₄ 3.0 g; d.H₂O to a total volume of 11

Mineral solution 2: KH₂PO₄-3.0 g; (NH₄)₂SO₄. 6.0 g; NaCl-6.0 g; MgSO₄. 0.6 g; CaCl₂-0.6 g; d. H₂O to a total volume of 11

Resazurin solution: 0.1% powdered resazurin in 100 ml distilled water.

Short chain fatty acid solution: Acetic acid −17 ml; Propionic acid-6 ml; n-Valeric acid-1 ml; Iso-Valeric acid-1 ml; Iso-Butyric acid—1 ml

Haemin solution: KOH-0.28 g Ethanol 95%-25 ml; Haemin-100 mg; d. H₂O to a total volume of 100 ml

Vitamin solution 1: Biotin-1 mg; Cobalamin-1 mg; p-Aminobenzoic acid-3 mg; Folic acid-5 mg; Pyridoxamine-15 mg; d. H₂O to a total volume of 100 ml

Vitamin solution 2: Thiamine-5 mg; Riboflavin-5 mg; d. H₂O to a total volume of 100 ml

Results

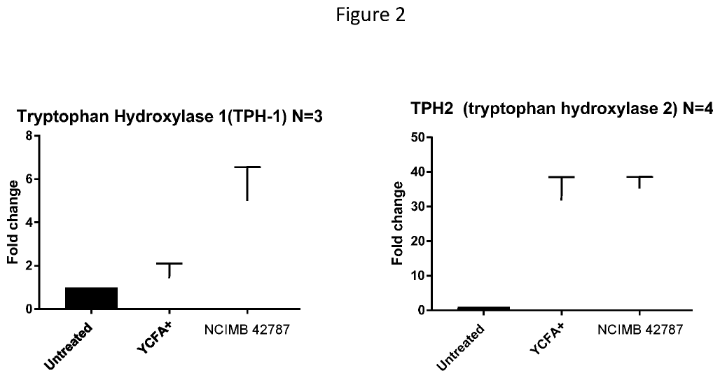

The results displayed in FIG. 2 show that when cells are incubated with 10% NCIMB 42787 bacterial cell-free supernatant for 24 h, the level of expression of TPH1 increases 5-fold, relative to untreated or YCFA⁺-treated controls. The level of expression of TPH2 also increases 30-fold relative to untreated controls.

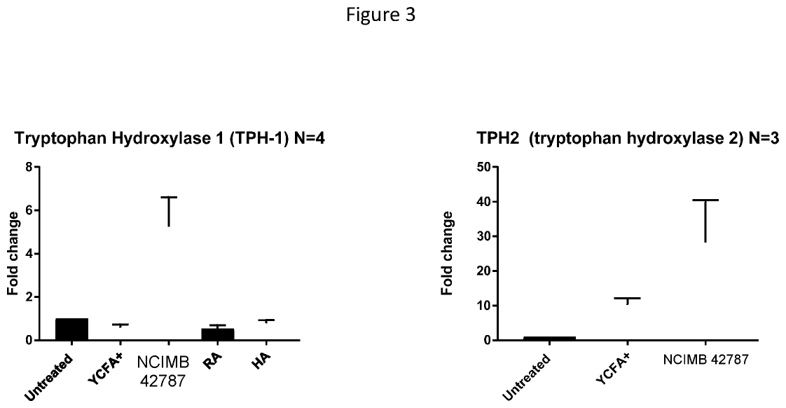

The results displayed in FIG. 3 show that when cells are incubated with 5% NCIMB 42787 bacterial cell-free supernatant for 72 h, the level of expression of TPH1 increases 5-fold, relative to untreated or YCFA^{m}-treated controls. The level of expression of TPH2 also increases 30-fold relative to untreated controls.

Compositions of the invention may therefore be useful for treating or preventing disorders characterised by serotonin deficiency by increasing the production of serotonin.

### Example 3—Serotonin Transporter Expression

Background

The SLC6A4 gene encodes serotonin transporter. Serotonin transporter is a biomarker of differentiated serotonergic neurons. The inventors thus sought to determine whether a bacterial strain of the species M. massilensis could upregulate serotonergic markers in neuron-like cells.

Material and Methods

Identical sets of experiments were carried out as described in Example 2. Primer sequences for the SLC6A4 gene are shown in Table 2.

TABLE 2
primer sequences for SLC6A4 and β-actin

| Gene | Forward | Reverse |
|---|---|---|
| SLC6A4 | AATCTGCCGATTTTCA | GTGTTGTAGTAGGAAGC |
| | AAG | AATG |
| | (SEQ ID NO: 7) | (SEQ ID NO: 8) |
| β-actin | GATCAAGATCATTGCT | TTGTCAAGAAAGGGTGT |
| | CCTC | AAC |
| | (SEQ ID NO: 5) | (SEQ ID NO: 6) |

Results

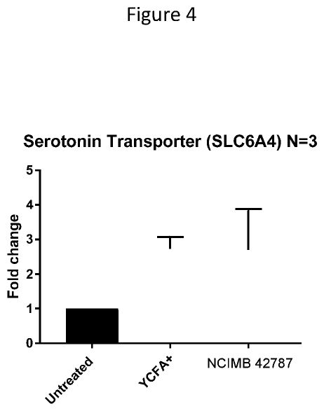

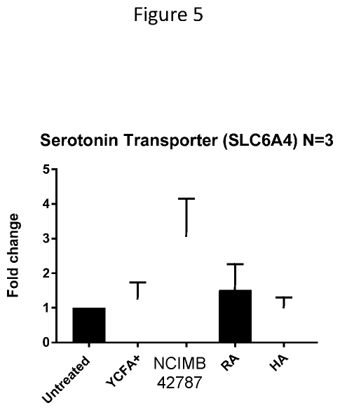

The results displayed in FIG. 4 shows that when cells are incubated with 10% Megasphaera massiliensis strain NCIMB 42787 bacterial cell-free supernatant for 24 h, the expression of SLC6A4 is upregulated 3-fold, relative to untreated controls but there was no difference with YCFA+ treated cells. The results displayed in FIG. 5 shows that when cells are incubated with 5% NCIMB 42787 bacterial cell-free supernatant for 72 h, the expression of SLC6A4 is upregulated 3-fold, relative to untreated controls and about 2-fold relative to YCFA+ treated cells.

Increasing the number of serotonergic neurons in vivo may explain how NCIMB 42787 increased the level of serotonin in the brain of healthy mice fed with NCIMB 42787. Compositions of the invention therefore may be useful for treating or preventing disorders characterised by serotonin deficiency.

### Example 4—Tryptophan Hydroxylase and Serotonin Transporter Expression Analysis in Caco2 Cells

Introduction

The majority of serotonin is produced in the gut. Gut serotonin is thought to play an important communicative role between the gut and the brain. Therefore, the inventors sought to determine whether Megasphaera massiliensis strain NCIMB 42787 could increase the expression of TPH1 and SLC6A4 in gut-like cells, thus indicating whether NCIMB 42787 could increase the level of serotonin in the gut, which may enhance serotonin bioavailability elsewhere, for example, in the brain.

To this end, the inventors incubated differentiated Caco2 cells with NCIMB 42787 bacterial cell-free supernatant. Differentiated Caco2 cells form polarized apical/mucosal and basolateral/serosal membranes that are impermeable and are structurally and functionally similar to epithelial cells of the small intestine.

Materials and Methods

Caco2 cells seeded on 12 well plates and differentiated for 10 days; then they were serum-starved for 12 hours and subsequently exposed to 10% supernatant derived from stationary phase NCIMB 42787 for 24 h. Cells were collected, and total RNA was isolated according to the RNeasy mini kit protocol (Qiagen). cDNA was made using the high capacity cDNA reverse transcription kit (Applied Biosystems). Gene expression was measured by qPCR. β-actin was used as internal control. Fold change was calculated according to the 2\hat{ }(−ΔΔct) method. Primer sequences are displayed in Table 1.

Results

The results displayed in FIG. 6 shows that when differentiated Caco2 cells are incubated with 10% NCIMB 42787 bacterial cell-free supernatant for 24 h, the expression of TPH1 is upregulated almost 3-fold, relative to untreated and YCFA⁺-treated controls. The results displayed in FIG. 7 shows that the incubation increases the expression of SLC6A4 more than 3-fold, relative to untreated controls.

Increasing the level of serotonin in the gut may explain how NCIMB 42787 increased the level of serotonin in the brain in mice fed with NCIMB 42787.

### Example 5—Megasphaera massiliensis Strain NCIMB 42787 Metabolite Analysis

Introduction

The gut microbiota, with its immense diversity and metabolic capacity, represents a huge metabolic reservoir for production of a vast variety of molecules. Metabolites present in bacteria supernatants can directly influence the host response to oxidative stress, cell-to-cell communication and effect neuronal processes. The inventors sought to determine what metabolites, in particular what short chain fatty acids and medium chain fatty acids, are produced by the M massiliensis strain NCIMB 42787.

Material and Methods

Bacterial Culture and Cell-Free Supernatant Collection

Pure cultures of bacteria were grown anaerobically in YCFA broth until they reached their stationary growth phase. Cultures were centrifuged at 5,000×g for 5 minutes and the cell-free supernatant (CFS) was filtered using a 0.2 μM filter (Millipore, UK). 1 mL aliquots of the CFS were stored at −80° C. until use. Sodium butyrate, hexanoic and valeric acid were obtained from Sigma Aldrich (UK) and suspensions were prepared in YCFA+ broth.

SCFA and MCFA Quantification of Bacterial Supernatants

Short chain fatty acids (SCFAs) and medium chain fatty acids (MCFAs) from bacterial supernatants were analysed and quantified by MS Omics APS as follows. Samples were acidified using hydrochloride acid, and deuterium labelled internal standards where added. All samples were analysed in a randomized order. Analysis was performed using a high polarity column (Zebron™ ZB-FFAP, GC Cap. Column 30 m×0.25 mm×0.25 μm) installed in a GC (7890B, Agilent) coupled with a quadropole detector (59977B, Agilent). The system was controlled by ChemStation (Agilent). Raw data was converted to netCDF format using Chemstation (Agilent), before the data was imported and processed in Matlab R2014b (Mathworks, Inc.) using the PARADISe software described in [50].

Results

NCIMB 42787 Produces Metabolites Butyrate and Valeric Acid

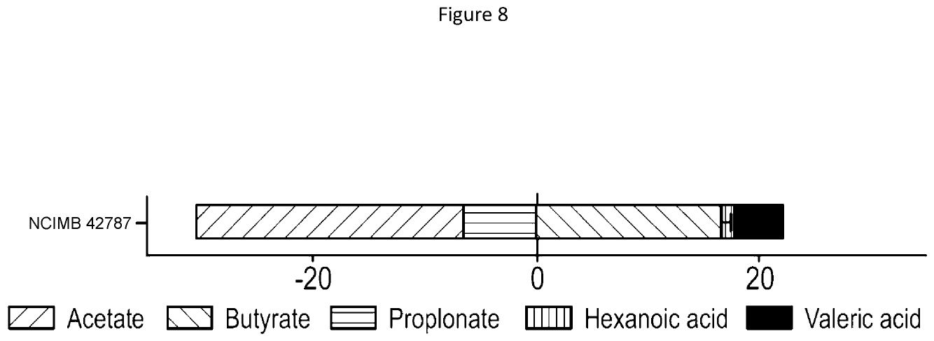

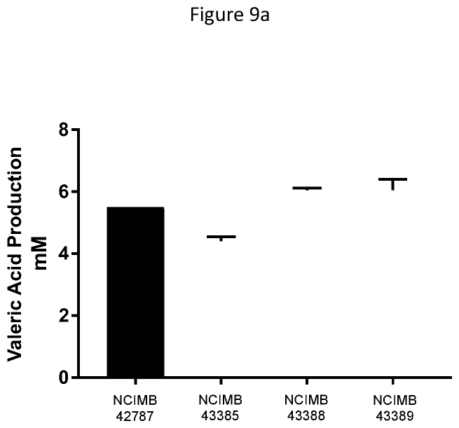

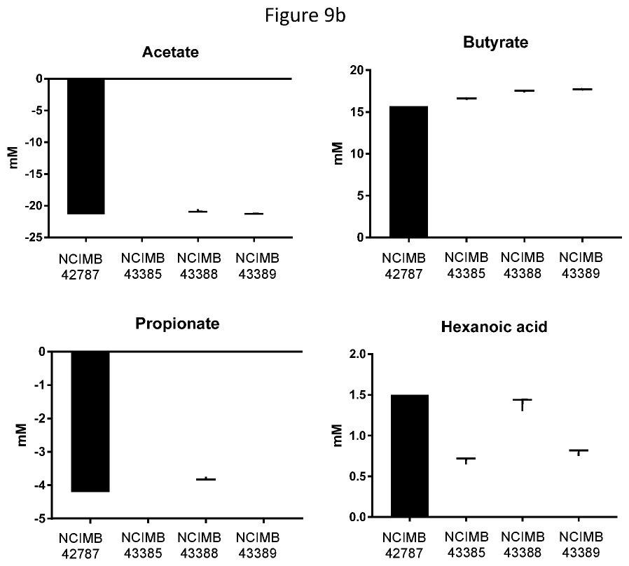

NCIMB 42787 produced valeric acid, hexanoic acid, at mean concentrations of 5.08 mM, 1.60 mM, respectively (FIG. 8). The inventors also found other strains of the species M. massiliensis produces comparable levels of valeric acid, hexanoic acid, butyric acid and consume similar amounts of acetate and propionate (FIG. 9).

The production of butyrate may be significant in the context of increasing serotonin secretion in the brain, because butyrate has a known role is reducing impermeability of the blood brain barrier, meaning bioactive molecules that enhance serotonin secretion have increased access to the brain when NCIMB 42787 is present [51]. This property of NCIMB 42787 (and other bacteria) may contribute to its efficacy.

### Example 6—Stability Testing

A composition described herein containing at least one bacterial strain described herein is stored in a sealed container at 25° C. or 4° C. and the container is placed in an atmosphere having 30%, 40%, 50%, 60%, 70%, 75%, 80%, 90% or 95% relative humidity. After 1 month, 2 months, 3 months, 6 months, 1 year, 1.5 years, 2 years, 2.5 years or 3 years, at least 50%, 60%, 70%, 80% or 90% of the bacterial strain shall remain as measured in colony forming units determined by standard protocols.

### Example 7-Metabolite Analysis

Further to the data provided in Example 5, FIG. 10 demonstrates what other short chain fatty acids are produced and consumed by the M. massiliensis strain NCIMB 42787 and strains deposited under accession numbers NCIMB 43385, NCIMB 43388 and NCIMB 43389.

M. massiliensis strain NCIMB 42787 reduces formic acid while increasing levels of 2-methyl-propanoic and 3-methyl-propanoic acid (FIG. 10). Therefore, strain NCIMB 42787 produces 2-methyl-propanoic and 3-methyl-propanoic acid and consumes formic acid. The inventors also found that other deposited strains produce comparable levels of 2-methyl-propanoic and 3-methyl-propanoic acid and consume similar amounts of formic acid.

### Example 8—NCIMB 42787 Causes a Significant Increase in Tph1 mRNA Expression in the Colon of BALB/c Mice

BALB/c mice were administered live biotherapeutic and tissues were isolated for analysis of gene expression using qPCR.

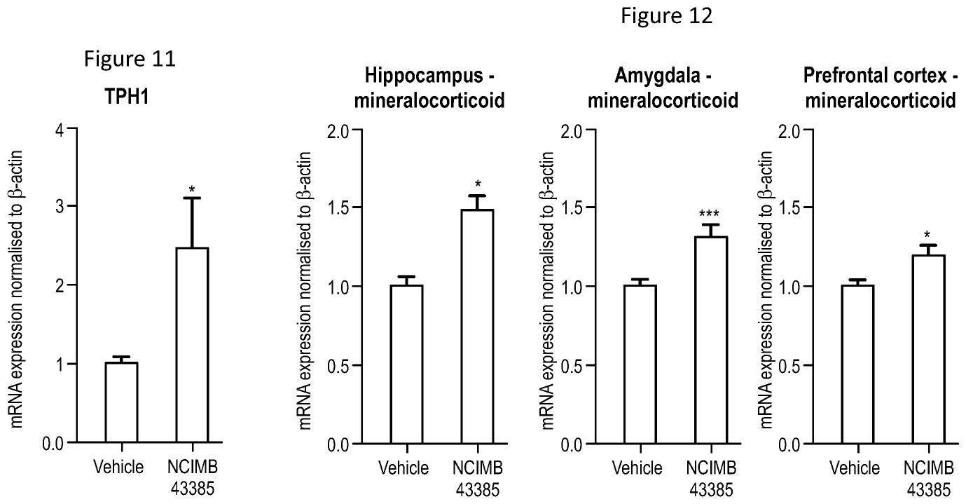

FIG. 11 demonstrates that NCIMB 42787 triggers increased expression of Tph1 in the colon of BALB/c mice (using quantification by qPCR normalised to β-actin) compared to the vehicle control.

As outlined above, Tph1 drives the production of serotonin and therefore, increases in Tph1 would be expected to be effective in the treatment of depression. In certain embodiments, the compositions of the invention increase the expression of Tph1. In certain embodiments, the compositions of the invention increase serotonin production via an increase in Tph1 expression. In certain embodiments, the compositions of the invention are therapeutically effective, in particular in depression, by driving the increase in serotonin expression.

### Example 9—Megasphaera Strain NCIMB 43385 Significantly Increases Mineralocorticoid Receptor Expression in the Hippocampus, Amygdala and Prefrontal Cortext of BALB/c Mice

BALB/c mice were administered live biotherapeutic and tissues were isolated for analysis of gene expression using qPCR.

FIG. 12 demonstrates the ability of NCIMB 43385 to significantly increase mineralocorticoid receptor expression in three areas of the brain (using quantification by qPCR normalised to β-actin) compared to the vehicle control.

It is understood that depression can be accompanied by decreased mineralocorticoid activity. Indeed, this receptor is seen to be down-regulated by chronic stress and during depression, but induced by anti-depressants. Increased mineralocorticoid receptor activity inhibits HPA axis activity and can have therapeutically beneficial effects, including promoting slow wave sleep, reducing anxiety and switching circuit connectivity to support copying. In certain embodiments, the compositions of the present invention increase expression of the mineralocorticoid receptor. In certain embodiments, the compositions of the present invention display therapeutic efficacy in depression and other disorders associated with serotonin deficiency in light of the impact on mineralocorticoid receptor expression.

### Example 10—Megasphaera Strain NCIMB 43385 Significantly Increases GABA A2 Expression in the Hippocampus and Amygdala of BALB/c Mice

BALB/c mice were administered live biotherapeutic and tissues were isolated for analysis of gene expression using qPCR.

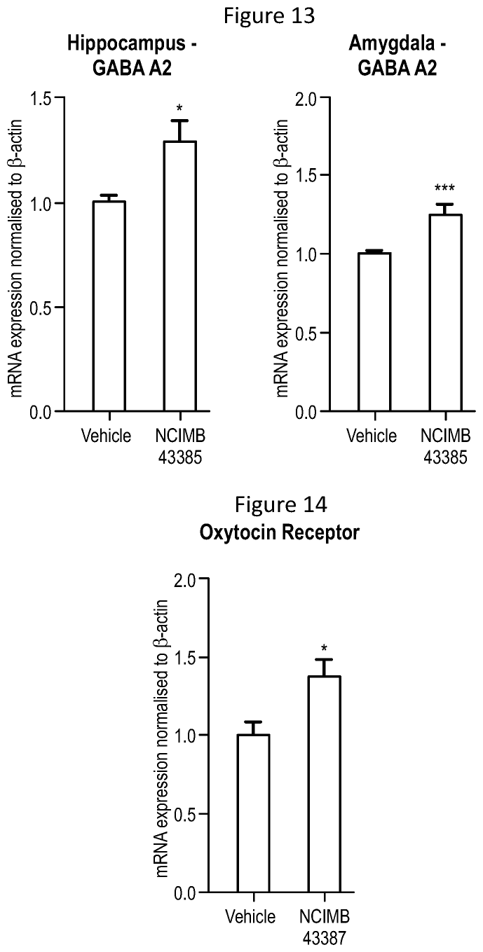

FIG. 13 demonstrates the ability of NCIMB 43385 to significantly increase GABA A2 expression in the brain (using quantification by qPCR normalised to β-actin) compared to the vehicle control.

The neurotransmitter GABA has been implicated to play a role in depression and GABA levels are reduced in patients with major depression disorder and are normalised following chronic treatment with antidepressants. Accordingly, increasing expression of GABA A2 has beneficial therapeutic effects in disorders such as depression and anxiety. In certain embodiments, the compositions of the present invention increase expression of GABA A2. In certain embodiments, the compositions of the present invention demonstrate therapeutic efficacy in disorders associated with serotonin deficiency, for example depression, in light of the increase in expression of GABA A2.

### Example 11—Megasphaera Strain NCIMB 43387 Significantly Increases Oxytocin Receptor Expression in the Prefrontal Cortex of BALB/c Mice

BALB/c mice were administered live biotherapeutic and tissues were isolated for analysis of gene expression using qPCR.

FIG. 14 demonstrates that NCIMB 43387 triggers a significant increase in expression of oxytocin receptor in the prefrontal cortex (using quantification by qPCR normalised to 0-actin) compared to the vehicle control.

Oxytocin is associated with emotional, social, cognitive and neuroendocrine physiologies as well as autoregulation. In particular, oxytocin release is involved in anxiolysis; positive mood; maternal behaviour, pair bonding; sexual behaviour; social memory; olfactory memory; anorexiant effects; attenuation of the HPA axis response to stress; autoexcitation during birth and suckling as well as other physiological and psychological processes. In certain embodiments, the compositions of the invention increase the levels of oxytocin. In certain embodiments, the compositions of the invention a therapeutically beneficial in disorders associated with serotonin deficiency in light of the increase in expression of the oxytocin receptor.

### Example 12—Effect of Megasphaera massiliensis Strain DSM 26228, Megasphaera elsdenii Strain NCIMB 8927 and Megasphaera massiliensis Strain NCIMB 42787 on Short-Chain Fatty Acid Production In Vitro

Summary

This study investigated the effect of DSM 26228, NCIMB 8927 and NCIMB 42787 on the production of short-chain fatty acids (SCFAs) in vitro. SCFAs, which include acetate, propionate, valerate, isobutyrate and isovalerate are microbial by-products of dietary fibre. An increase in any SCFA suggests an increase in productivity of the microbiota and is a desirable trait.

Material and Methods

Pure cultures of DSM 26228, NCIMB 8927 and NCIMB 42787 were grown anaerobically in YCFA+ broth [Per litre: Casein hydrolysate 10.0 g, Yeast Extract 2.5 g, Sodium hydrogen carbonate 4.0 g, Glucose 2.0 g, Cellobiose 2.0 g, Soluble starch 2.0 g, Di-potassium hydrogen phosphate 0.45 g, Potassium di-hydrogen phosphate 0.45 g, Resazurin 0.001 g, L-Cysteine HCl 1.0 g, Ammonium sulphate 0.9 g, Sodium chloride 0.9 g, Magnesium sulphate 0.09 g, Calcium chloride 0.09 g, Haemin 0.01 g, SCFA 3.1 ml (Acetic acid 2.026 ml/L, Propionic acid 0.715 ml/L, n-Valeric acid 0.119 ml/L, Iso-Valeric acid 0.119 ml/L, Iso-Butyric acid 0.119 ml/L), vitamin mix 1:1 ml (Biotin 1 mg/100 ml, Cyanocobalamine 1 mg/100 ml, p-Aminobenzoic acid 3 mg/100 ml, Pyridoxine 15 mg/100 ml), vitamin mix 2:1 ml (Thiamine 5 mg/100 ml, Riboflavin 5 mg/100 ml), vitamin mix 3:1 ml (Folic acid 5 mg/100 ml)] until they reached their stationary growth phase. Cultures were centrifuged at 5000×g for 10 minutes and the cell-free supernatant (CFS) was filtered using a 0.45 μM followed by a 0.2 μM filter (Millipore, UK), after which 1 mL aliquots of the CFS were stored at −80° C. until use.

Short chain fatty acids (SCFAs) and medium chain fatty acids (MCFAs) from bacterial supernatants were analysed and quantified by MS Omics APS, Denmark. Samples were acidified using hydrochloride acid, and deuterium labelled internal standards were added. All samples were analyzed in a randomized order. Analysis was performed using a high polarity column (Zebron™ ZB-FFAP, GC Cap. Column 30 m×0.25 mm×0.25 m) installed in a gas chromatograph (7890B, Agilent) coupled with a quadropole detector (5977B, Agilent). The system was controlled by ChemStation (Agilent). Raw data was converted to netCDF format using Chemstation (Agilent), before the data was imported and processed in Matlab R2014b (Mathworks, Inc.) using the PARADISe software described by reference [50].

Results

The following pattern was observed for each of the bacterial strains:

| | | | | 2-methyl- | | 3-methyl- | | 4-methyl- | | |
|---|---|---|---|---|---|---|---|---|---|---|
| | Acetic | Formic | Propanoic | propanoic | Butanoic | butanoic | Pentanoic | pentanoic | Hexanoic | Heptanoic |
| | acid | acid | acid | acid | acid | acid | acid | acid | acid | acid |
| DSM | −17.6 | −0.4 | −4.9 | 1.7 | 16.0 | 1.5 | 5.8 | 0.0 | 1.6 | 0.1 |
| 26228 | | | | | | | | | | |
| NCIMB | −2.4 | −0.1 | −2.5 | 0.2 | 10.7 | 0.5 | 2.8 | 0.0 | 0.3 | 0.1 |
| 8927 | | | | | | | | | | |
| NCIMB | −20.2 | −0.4 | −5.6 | 2.1 | 15.8 | 4.5 | 6.6 | 0.0 | 2.2 | 0.1 |
| 42787 | | | | | | | | | | |

These data demonstrate that all three strains of Megasphaera increase butyrate (butanoic acid) and valeric acid (pentanoic acid).

Valeric acid is a GABA-A agonist and is thought to increase the availability of synaptic GABA and/or enhance postsynaptic GABA responses, thus enhancing GABAergic activity. Accordingly, as outlined above, increasing the GABA response has beneficial therapeutic effects in disorders such as depression and anxiety. Both Megasphaera massiliensis and Megasphaera elsdenii strains trigger beneficial increases in valeric acid. Therefore, in certain embodiments, the compositions of the present invention demonstrate therapeutic efficacy in disorders associated with serotonin deficiency, for example depression, in light of the increase in valeric acid.

As outlined above, butyrate has neuroprotective activity, increases serotonin and BDNF expression and can assist in restoration of blood brain barrier impairments. Accordingly, butyrate can significantly improve depression-like behaviours. Both Megasphaera massiliensis and Megasphaera elsdenii strains trigger beneficial increases in butyrate. In certain embodiments, the compositions of the present invention demonstrate therapeutic efficacy in disorders associated with serotonin deficiency, for example depression, in light of the increase in butyrate.

### Example 13—Short/Medium Chain Fatty Acid Production Profile of Megasphaera massiliensis Strain NCIMB 43389 and Megasphaera sp. Strain NCIMB 43385

Materials and Methods

Pure cultures of Megasphaera massiliensis strain NCIMB 43389 and Megasphaera sp. strain NCIMB 43385 were grown anaerobically in YCFA+ broth. Short chain fatty acids (SCFAs) and medium chain fatty acids (MCFAs) from bacterial supernatants were analysed and quantified by MS Omics APS, Denmark. Samples were acidified using hydrochloride acid, and deuterium labelled internal standards were added. All samples were analyzed in a randomized order. Analysis was performed using a high polarity column (Zebron™ ZB-FFAP, GC Cap. Column 30 m×0.25 mm×0.25 m) installed in a gas chromatograph (7890B, Agilent) coupled with a quadropole detector (5977B, Agilent). The system was controlled by ChemStation (Agilent). Raw data was converted to netCDF format using Chemstation (Agilent), before the data was imported and processed in Matlab R2014b (Mathworks, Inc.) using the PARADISe software.

Results

| | Change in short/medium chain fatty acid concentration (mM) | | | | | | |
|---|---|---|---|---|---|---|---|
| | Succinic | Formic | Acetic | Propionic | Butyric | Valeric | Hexanoic |
| Strain | acid | acid | acid | acid | acid | acid | acid |
| Megasphaera | Not | 1.51 | −16 | −5.25 | 27.86 | 6.44 | 0.96 |
| massthensis NCIMB | detected | | | | | | |
| 43389 | | | | | | | |
| Megasphaera sp. | Not | Not | 7.73 | −1.07 | 16.28 | 5.94 | 1.22 |
| strain NCIMB 43385 | detected | detected | | | | | |

The results are consistent with those reported in Example 7 (and FIGS. 9-10) for Megasphaera massiliensis strain NCIMB 43389 and Megasphaera sp. strain NCIMB 43385, further confirming the short/medium fatty acid chain profile of these strains.

Sequences

(Megasphaera massiliensis gene for 16S ribosomal RNA, partial sequence,
strain: NP3-JX424772.1)
SEQ ID NO: 1

| | |
|---|---|
| 1 | agagtttgat cctggctcag gacgaacgct ggcggcgtgc ttaacacatg caagtcgaac |
| 61 | gagaagagat gagaagcttg cttcttatca attcgagtgg caaacgggtg agtaacgcgt |
| 121 | aagcaacctg cccttcagat ggggacaaca gctggaaacg gctgctaata ccgaatacgt |
| 181 | tctttccgcc gcatgacggg aagaagaaag ggaggccttc gggctttcgc tggaggaggg |
| 241 | gcttgcgtct gattagctag ttggaggggt aacggcccac caaggcgacg atcagtagcc |
| 301 | ggtctgagag gatgaacggc cacattggga ctgagacacg gcccagactc ctacgggagg |
| 361 | cagcagtggg gaatcttccg caatggacga aagtctgacg gagcaacgcc gcgtgaacga |
| 421 | tgacggcctt cgggttgtaa agttctgtta tatgggacga acagggcatc ggttaatacc |
| 481 | cggtgtcttt gacggtaccg taagagaaag ccacggctaa ctacgtgcca gcagccgcgg |
| 541 | taatacgtag gtggcaagcg ttgtccggaa ttattgggcg taaagggcgc gcaggcggca |
| 601 | tcgcaagtcg gtcttaaaag tgcggggctt aaccccgtga ggggaccgaa actgtgaagc |
| 661 | tcgagtgtcg gagaggaaag cggaattcct agtgtagcgg tgaaatgcgt agatattagg |
| 721 | aggaacacca gtggcgaaag cggctttctg gacgacaact gacgctgagg cgcgaaagcc |
| 781 | aggggagcaa acgggattag ataccccggt agtcctggcc gtaaacgatg gatactaggt |
| 841 | gtaggaggta tcgactcctt ctgtgccgga gttaacgcaa taagtatccc gcctggggag |
| 901 | tacggccgca aggctgaaac tcaaaggaat tgacgggggc ccgcacaagc ggtggagtat |
| 961 | gtggtttaat tcgacgcaac gcgaagaacc ttaccaagcc ttgacattga ttgctacgga |
| 1021 | aagagatttc cggttcttct tcggaagaca agaaaacagg tggtgcacgg ctgtcgtcag |
| 1081 | ctcgtgtcgt gagatgttgg gttaagtccc gcaacgagcg caacccctat cttctgttgc |
| 1141 | cagcacctcg ggtggggact cagaagagac tgccgcagac aatgcggagg aaggcgggga |
| 1201 | tgacgtcaag tcatcatgcc ccttatggct tgggctacac acgtactaca atggctctta |
| 1261 | atagagggac gcgaaggagc gatccggagc aaaccccaaa aacagagtcc cagttcggat |
| 1321 | tgcaggctgc aactcgcctg catgaagcag gaatcgctag taatcgcagg tcagcatact |
| 1381 | gcggtgaata cgttcccggg ccttgtacac accgcccgtc acaccacgaa agtcattcac |
| 1441 | acccgaagcc ggtgaggcaa ccgcaaggaa ccagccgtcg aaggtggggg cgatgattgg |
| 1501 | ggtgaagtcg taacaaggt |

(consensus 16S rRNA gene sequence for Megasphaera massiliensis
strain NCIMB 42787)
SEQ ID NO: 2
TGAGAAGCTTGCTTCTTATCGATTCTAGTGGCAAACGGGTGAGTAACGCGTAAGCAACC
TGCCCTTCAGATGGGGACAACAGCTGGAAACGGCTGCTAATACCGAATACGTTCTTTCC
GCCGCATGACGGGAAGAAGAAAGGGAGGCCTTCGGGCTTTCGCTGGAGGAGGGGCTTG
CGTCTGATTAGCTAGTTGGAGGGGTAACGGCCCACCAAGGCGACGATCAGTAGCCGGTC
TGAGAGGATGAACGGCCACATTGGGACTGAGACACGGCCCAGACTCCTACGGGAGGCA
GCAGTGGGGAATCTTCCGCAATGGACGAAAGTCTGACGGAGCAACGCCGCGTGAACGA
TGACGGCCTTCGGGTTGTAAAGTTCTGTTATATGGGACGAACAGGACATCGGTTAATAC
CCGGTGTCTTTGACGGTACCGTAAGAGAAAGCCACGGCTAACTACGTGCCAGCAGCCGC
GGTAATACGTAGGTGGCAAGCGTTGTCCGGAATTATTGGGCGTAAAGGGCGCGCAGGC
GGCATCGCAAGTCGGTCTTAAAAGTGCGGGGCTTAACCCCGTGAGGGGACCGAAACTGT
GAAGCTCGAGTGTCGGAGAGGAAAGCGGAATTCCTAGTGTAGCGGTGAAATGCGTAGA
TATTAGGAGGAACACCAGTGGCGAAAGCGGCTTTCTGGACGACAACTGACGCTGAGGC
GCGAAAGCCAGGGGAGCAAACGGGATTAGATACCCCGGTAGTCCTGGCCGTAAACGAT
GGATACTAGGTGTAGGAGGTATCGACTCCTTCTGTGCCGGAGTTAACGCAATAAGTATC
CCGCCTGGGGAGTACGGCCGCAAGGCTGAAACTCAAAGGAATTGACGGGGGCCCGCAC
AAGCGGTGGAGTATGTGGTTTAATTCGACGCAACGCGAAGAACCTTACCAAGCCTTGAC
ATTGATTGCTACGGAAAGAGATTTCCGGTTCTTCTTCGGAAGACAAGAAAACAGGTGGT
GCACGGCTGTCGTCAGCTCGTGTCGTGAGATGTTGGGTTAAGTCCCGCAACGAGCGCAA
CCCCTATCTTCTGTTGCCAGCACCTCGGGTGGGGACTCAGAAGAGACTGCCGCAGACAA
TGCGGAGGAAGGCGGGGATGACGTCAAGTCATCATGCCCCTTATGGCTTGGGCTACACA
CGTACTACAATGGCTCTTAATAGAGGGAAGCGAAGGAGCGATCCGGAGCAAACCCCAA
AAACAGAGTCCCAGTTCGGATTGCAGGCTGCAACTCGCCTGCATGAAGCAGGAATCGCT
AGTAATCGCAGGTCAGCATACTGCGGTGAATACGTTCCCGGGCCTTGTACACACCGCCC
GTCACACCACGAAAGTCATTCACACCCGAAGCCGGTGAGGCAACCGCAAG

| Gene | Forward | Reverse |
|---|---|---|
| TPH1 | AAAGAGCGTACAGGTTTTTC | GTCTCACATATTGAGTGCAG |
| | (SEQ ID NO: 3) | (SEQ ID NO: 4) |
| β-actin | GATCAAGATCATTGCTCCTC | TTGTCAAGAAAGGGTGTAAC |
| | (SEQ ID NO: 5) | (SEQ ID NO: 6) |
| SLC6A4 | AATCTGCCGATTTTCAAAG | GTGTTGTAGTAGGAAGCAATG |
| | (SEQ ID NO: 7) | (SEQ ID NO: 8) |
| TPH2 | CACTATTGTGACGCTGAATC | AGCTCAGAACCATACATGAG |
| | (SEQ ID NO: 9) | (SEQ ID NO: 10) |

(consensus 16S rRNA sequence for the Megasphaera strain deposited under
accession number NCIMB 43385)
SEQ ID NO: 11
GGCTGGTTCCTTGCGGTTGCCTCACCGGCTTCGGGTGTGAATGACTTTCGTGGTGTGACG
GGCGGTGTGTACAAGGCCCGGGAACGTATTCACCGCAGTATGCTGACCTGCGATTACTA
GCGATTCCTGCTTCATGCAGGCGAGTTGCAGCCTGCAATCCGAACTGGGACTCTGTTTTT
GGGGTTTGCTCCGGATCGCTCCTTCGCTTCCCTCTATTAAGAGCCATTGTAGTACGTGTG
TAGCCCAAGCCATAAGGGGCATGATGACTTGACGTCATCCCCGCCTTCCTCCGCATTGTC
TGCGGCAGTCTCTTCTGAGTCCCCACCCTTAGTGCTGGCAACAGAAGATAGGGGTTGCG
CTCGTTGCGGGACTTAACCCAACATCTCACGACACGAGCTGACGACAGCCGTGCACCAC
CTGTTTTCTTGTCTTCCGAAGAAGAACCGGAAATCTCTTTCCGTAGCAATCAATGTCAAG
GCTTGGTAAGGTTCTTCGCGTTGCGTCGAATTAAACCACATACTCCACCGCTTGTGCGGG
CCCCCGTCAATTCCTTTGAGTTTCAGCCTTGCGGCCGTACTCCCCAGGCGGGATACTTAT
TGCGTTAACTCCGGCACAGAAGGAGTCGATACCTCCTACACCTAGTATCCATCGTTTACG
GCCAGGACTACCGGGGTATCTAATCCCGTTTGCTCCCCTGGCTTTCGCGCCTCAGCGTCA
GTTGTCGTCCAGAAAGCCGCTTTCGCCACTGGTGTTCCTCCTAATATCTACGCATTTCAC
CGCTACACTAGGAATTCCGCTTTCCTCTCCGACACTCGAGCTTCACAGTTTCGGTCCCCT
CACGGGGTTAAGCCCCGCACTTTTAAGACCGACTTGCGATGCCGCCTGCGCGCCCTTTAC
GCCCAATAATTCCGGACAACGCTTGCCACCTACGTATTACCGCGGCTGCTGGCACGTAG
TTAGCCGTGGCTTTCTCTTACGGTACCGTCAGGGATAACGGGTATTGACCGCTATCCTGT
TCGTCCCATATAACAGAACTTTACAACCCGAAGGCCGTCATCGTTCACGCGGCGTTGCTC
CGTCAGACTTTCGTCCATTGCGGAAGATTCCCCACTGCTGCCTCCCGTAGGAGTCTGGGC
CGTGTCTCAGTCCCAATGTGGCCGTTCATCCTCTCAGACCGGCTACTGATCGTCGCCTTG
GTGGGCCGTTACCCCTCCAACTAGCTAATCAGACGCAAGCCCCTCCTCCAGCGAAAGCC
CGAAGGCCTCCCTTTCTTCATCCCGTCATGCGGCGGAAAGAACGTATTCGGTATTAGCA
GCCGTTTCCAGCTGTTGTCCCCATCTGAAGGGCAGGTTGCTTACGCGTTACTCACCCGTT
TGCCACTCGAATTGATAAGAAGCAAGCTTCTCATC
(consensus 16S rRNA sequence for the Megasphaera massiliensis strain deposited
under accession number NCIMB 43388)
SEQ ID NO: 12
GGCTGGTTCCTTGCGGTTGCCTCACCGGCTTCGGGTGTGAATGACTTTCGTGGTGTGACG
GGCGGTGTGTACAAGGCCCGGGAACGTATTCACCGCAGTATGCTGACCTGCGATTACTA
GCGATTCCTGCTTCATGCAGGCGAGTTGCAGCCTGCAATCCGAACTGGGACTCTGTTTTT
GGGGTTTGCTCCGGATCGCTCCTTCGCTTCCCTCTATTAAGAGCCATTGTAGTACGTGTG
TAGCCCAAGCCATAAGGGGCATGATGACTTGACGTCATCCCCGCCTTCCTCCGCATTGTC
TGCGGCAGTCTCTTCTGAGTCCCCACCCGAGGTGCTGGCAACAGAAGATAGGGGTTGCG
CTCGTTGCGGGACTTAACCCAACATCTCACGACACGAGCTGACGACAGCCGTGCACCAC
CTGTTTTCTTGTCTTCCGAAGAAGAACCGGAAATCTCTTTCCGTAGCAATCAATGTCAAG
GCTTGGTAAGGTTCTTCGCGTTGCGTCGAATTAAACCACATACTCCACCGCTTGTGCGGG
CCCCCGTCAATTCCTTTGAGTTTCAGCCTTGCGGCCGTACTCCCCAGGCGGGATACTTAT
TGCGTTAACTCCGGCACAGAAGGAGTCGATACCTCCTACACCTAGTATCCATCGTTTACG
GCCAGGACTACCGGGGTATCTAATCCCGTTTGCTCCCCTGGCTTTCGCGCCTCAGCGTCA
GTTGTCGTCCAGAAAGCCGCTTTCGCCACTGGTGTTCCTCCTAATATCTACGCATTTCAC
CGCTACACTAGGAATTCCGCTTTCCTCTCCGACACTCGAGCTTCACAGTTTCGGTCCCCT
CACGGGGTTAAGCCCCGCACTTTTAAGACCGACTTGCGATGCCGCCTGCGCGCCCTTTAC
GCCCAATAATTCCGGACAACGCTTGCCACCTACGTATTACCGCGGCTGCTGGCACGTAG
TTAGCCGTGGCTTTCTCTTACGGTACCGTCAAAGACACCGGGTATTAACCGATGTCCTGT
TCGTCCCATATAACAGAACTTTACAACCCGAAGGCCGTCATCGTTCACGCGGCGTTGCTC
CGTCAGACTTTCGTCCATTGCGGAAGATTCCCCACTGCTGCCTCCCGTAGGAGTCTGGGC
CGTGTCTCAGTCCCAATGTGGCCGTTCATCCTCTCAGACCGGCTACTGATCGTCGCCTTG
GTGGGCCGTTACCCCTCCAACTAGCTAATCAGACGCAAGCCCCTCCTCCAGCGAAAGCC
CGAAGGCCTCCCTTTCTTCTTCCCGTCATGCGGCGGAAAGAACGTATTCGGTATTAGCAG
CCGTTTCCAGCTGTTGTCCCCATCTGAAGGGCAGGTTGCTTACGCGTTACTCACCCGTTT
GCCACTAGAATCGATAAGAAGCAAGCTTCTCATGTCTTCT
(consensus 16S rRNA sequence for the Megasphaera massilliensis strain deposited
under accession number NCIMB 43389)
SEQ ID NO: 13
CGACGGCTGGTTCCTTGCGGTTGCCTCACCGGCTTCGGGTGTGAATGACTTTCGTGGTGT
GACGGGCGGTGTGTACAAGGCCCGGGAACGTATTCACCGCAGTATGCTGACCTGCGATT
ACTAGCGATTCCTGCTTCATGCAGGCGAGTTGCAGCCTGCAATCCGAACTGGGACTCTG
TTTTTGGGGTTTGCTCCGGATCGCTCCTTCGCTTCCCTCTATTAAGAGCCATTGTAGTACG
TGTGTAGCCCAAGCCATAAGGGGCATGATGACTTGACGTCATCCCCGCCTTCCTCCGCAT
TGTCTGCGGCAGTCTCTTCTGAGTCCCCACCCGAGGTGCTGGCAACAGAAGATAGGGGT
TGCGCTCGTTGCGGGACTTAACCCAACATCTCACGACACGAGCTGACGACAGCCGTGCA
CCACCTGTTTTCTTGTCTTCCGAAGAAGAACCGGAAATCTCTTTCCGTAGCAATCAATGT
CAAGGCTTGGTAAGGTTCTTCGCGTTGCGTCGAATTAAACCACATACTCCACCGCTTGTG
CGGGCCCCCGTCAATTCCTTTGAGTTTCAGCCTTGCGGCCGTACTCCCCAGGCGGGATAC
TTATTGCGTTAACTCCGGCACAGAAGGAGTCGATACCTCCTACACCTAGTATCCATCGTT
TACGGCCAGGACTACCGGGGTATCTAATCCCGTTTGCTCCCCTGGCTTTCGCGCCTCAGC
GTCAGTTGTCGTCCAGAAAGCCGCTTTCGCCACTGGTGTTCCTCCTAATATCTACGCATT
TCACCGCTACACTAGGAATTCCGCTTTCCTCTCCGACACTCGAGCTTCACAGTTTCGGTC
CCCTCACGGGGTTAAGCCCCGCACTTTTAAGACCGACTTGCGATGCCGCCTGCGCGCCCT
TTACGCCCAATAATTCCGGACAACGCTTGCCACCTACGTATTACCGCGGCTGCTGGCAC
GTAGTTAGCCGTGGCTTTCTCTTACGGTACCGTCAAAGACACCGGGTATTAACCGATGCC
CTGTTCGTCCCATATAACAGAACTTTACAACCCGAAGGCCGTCATCGTTCACGCGGCGTT
GCTCCGTCAGACTTTCGTCCATTGCGGAAGATTCCCCACTGCTGCCTCCCGTAGGAGTCT
GGGCCGTGTCTCAGTCCCAATGTGGCCGTTCATCCTCTCAGACCGGCTACTGATCGTCGC
CTTGGTGGGCCGTTACCCCTCCAACCAGCTAATCAGACGCAAGCCCCTCCTCCAGCGAA
AGCCCGAAGGCCTCCCTTTCTTCTTCCCGTCATGCGGCGGAAAGAACGTATTCGGTATTA
GCAGCCGTTTCCAGCTGTTGTCCCCATCTGAAGGGCAGGTTGCTTACGCGTTACTCACCC
GTTTGCCACTAGAATCGATAAGAAGCAAGCTTCTCATGTCTTCTCGTTCGACTTGCAT
(consensus 16S rRNA sequence for the Megasphaera strain deposited under
accession number NCIMB 43386)
SEQ ID NO: 14
CGACGGCTGGTTCCTTGCGGTTGCCTCACCGGCTTCGGGTGTGAATGACTTTCGTGGTGT
GACGGGCGGTGTGTACAAGGCCCGGGAACGTATTCACCGCAGTATGCTGACCTGCGATT
ACTAGCGATTCCTGCTTCATGCAGGCGAGTTGCAGCCTGCAATCCGAACTGGGACTCTG
TTTTTGGGGTTTGCTCCGGATCGCTCCTTCGCTTCCCTCTATTAAGAGCCATTGTAGTACG
TGTGTAGCCCAAGCCATAAGGGGCATGATGACTTGACGTCATCCCCGCCTTCCTCCGCAT
TGTCTGCGGCAGTCTCTTCTGAGTCCCCACCCTTAGTGCTGGCAACAGAAGATAGGGGTT
GCGCTCGTTGCGGGACTTAACCCAACATCTCACGACACGAGCTGACGACAGCCGTGCAC
CACCTGTTTTCTTGTCTTCCGAAGAAGAACCGGAAATCTCTTTCCGTAGCAATCAATGTC
AAGGCTTGGTAAGGTTCTTCGCGTTGCGTCGAATTAAACCACATACTCCACCGCTTGTGC
GGGCCCCCGTCAATTCCTTTGAGTTTCAGCCTTGCGGCCGTACTCCCCAGGCGGGATACT
TATTGCGTTAACTCCGGCACAGAAGGAGTCGATACCTCCTACACCTAGTATCCATCGTTT
ACGGCCAGGACTACCGGGGTATCTAATCCCGTTTGCTCCCCTGGCTTTCGCGCCTCAGCG
TCAGTTGTCGTCCAGAAAGCCGCTTTCGCCACTGGTGTTCCTCCTAATATCTACGCATTT
CACCGCTACACTAGGAATTCCGCTTTCCTCTCCGACACTCGAGCTTCACAGTTTCGGTCC
CCTCACGGGGTTAAGCCCCGCACTTTTAAGACCGACTTGCGATGCCGCCTGCGCGCCCTT
TACGCCCAATAATTCCGGACAACGCTTGCCACCTACGTATTACCGCGGCTGCTGGCACG
TAGTTAGCCGTGGCTTTCTCTTACGGTACCGTCAGGGATAACGGGTATTGACCGCTATCC
TGTTCGTCCCATATAACAGAACTTTACAACCCGAAGGCCGTCATCGTTCACGCGGCGTTG
CTCCGTCAGACTTTCGTCCATTGCGGAAGATTCCCCACTGCTGCCTCCCGTAGGAGTCTG
GGCCGTGTCTCAGTCCCAATGTGGCCGTTCATCCTCTCAGACCGGCTACTGATCGTCGCC
TTGGTGGGCCGTTACCCCTCCAACTAGCTAATCAGACGCAAGCCCCTCCTCCAGCGAAA
GCCCGAAGGCCTCCCTTTCTTCATCCCGTCATGCGGCGGAAAGAACGTATTCGGTATTAG
CAGCCGTTTCCAGCTGTTGTCCCCATCTGAAGGGCAGGTTGCTTACGCGTTACTCACCCG
TTTGCCACTCGAATTGATAAGAAGCAAGCTTCTCATCTCTTCTCGTTCGACTGCA
(consensus 16S rRNA sequence for the Megasphaera strain deposited under accession
number NCIMB 43387)
SEQ ID NO: 15
TCGAACGGCTGGTTCCTTGCGGTTGCCTCACCGGCTTCGGGTGTGAATGACTTTCGTGGT
GTGACGGGCGGTGTGTACAAGGCCCGGGAACGTATTCACCGCAGTATGCTGACCTGCGA
TTACTAGCGATTCCTGCTTCATGCAGGCGAGTTGCAGCCTGCAATCCGAACTGGGACTCT
GTTTTTGGGGTTTGCTCCGGATCGCTCCTTCGCTTCCCTCTATTAAGAGCCATTGTAGTAC
GTGTGTAGCCCAAGCCATAAGGGGCATGATGACTTGACGTCATCCCCGCCTTCCTCCGC
ATTGTCTGCGGCAGTCTCTTCTGAGTCCCCACCCTTAGTGCTGGCAACAGAAGATAGGG
GTTGCGCTCGTTGCGGGACTTAACCCAACATCTCACGACACGAGCTGACGACAGCCGTG
CACCACCTGTTTTCTTGTCTTCCGAAGAAGAACCGGAAATCTCTTTCCGTAGCAATCAAT
GTCAAGGCTTGGTAAGGTTCTTCGCGTTGCGTCGAATTAAACCACATACTCCACCGCTTG
TGCGGGCCCCCGTCAATTCCTTTGAGTTTCAGCCTTGCGGCCGTACTCCCCAGGCGGGAT
ACTTATTGCGTTAACTCCGGCACAGAAGGAGTCGATACCTCCTACACCTAGTATCCATCG
TTTACGGCCAGGACTACCGGGGTATCTAATCCCGTTTGCTCCCCTGGCTTTCGCGCCTCA
GCGTCAGTTGTCGTCCAGAAAGCCGCTTTCGCCACTGGTGTTCCTCCTAATATCTACGCA
TTTCACCGCTACACTAGGAATTCCGCTTTCCTCTCCGACACTCGAGCTTCACAGTTTCGG
TCCCCTCACGGGGTTAAGCCCCGCACTTTTAAGACCGACTTGCGATGCCGCCTGCGCGCC
CTTTACGCCCAATAATTCCGGACAACGCTTGCCACCTACGTATTACCGCGGCTGCTGGCA
CGTAGTTAGCCGTGGCTTTCTCTTACGGTACCGTCAGGGATAACGGGTATTGACCGCTAT
CCTGTTCGTCCCATATAACAGAACTTTACAACCCGAAGGCCGTCATCGTTCACGCGGCGT
TGCTCCGTCAGACTTTCGTCCATTGCGGAAGATTCCCCACTGCTGCCTCCCGTAGGAGTC
TGGGCCGTGTCTCAGTCCCAATGTGGCCGTTCATCCTCTCAGACCGGCTACTGATCGTCG
CCTTGGTGGGCCGTTACCCCTCCAACTAGCTAATCAGACGCAAGCCCCTCCTCCAGCGA
AAGCCCGAAGGCCTCCCTTTCTTCATCCCGTCATGCGGCGGAAAGAACGTATTCGGTATT
AGCAGCCGTTTCCAGCTGTTGTCCCCATCTGAAGGGCAGGTTGCTTACGCGTTACTCACC
CGTTTGCCACTCGAATTGATAAGAAGCAAGCTTCTCATCTCTTCTCGTTCGACTTGCA
(consensus 16S rRNA sequence for the M. elsdenii strain deposited under accession
number NCIMB 8927)
SEQ ID NO: 16

| | |
|---|---|
| 1 | agagtttgat cctggctcag gacgaacgct ggcggcgtgc ttaacacatg caagtcgaac |
| 61 | gagaagagat gagaagcttg cttcttatcg attcgagtgg caaacgggtg agtaacgcgt |
| 121 | aagcaacctg cccttcagat ggggacaaca gctggaaacg gctgctaata ccgaatacgt |
| 181 | tctttttgtc gcatggcaga gggaagaaag ggaggctctt cggagctttc gctgaaggag |
| 241 | gggcttgcgt ctgattagct agttggaggg gtaacggccc accaaggcga cgatcagtag |
| 301 | ccggtctgag aggatgaacg gccacattgg gactgagaca cggcccagac tcctacggga |
| 361 | ggcagcagtg gggaatcttc cgcaatggac gaaagtctga cggagcaacg ccgcgtgaac |
| 421 | gatgacggcc ttcgggttgt aaagttctgt tatacgggac gaatggcgta gcggtcaata |
| 481 | cccgttacga gtgacggtac cgtaagagaa agccacggct aactacgtgc cagcagccgc |
| 541 | ggtaatacgt aggtggcaag cgttgtccgg aattattggg cgtaaagggc gcgcaggcgg |
| 601 | cgtcgtaagt cggtcttaaa agtgcggggc ttaaccccgt gaggggaccg aaactgcgat |
| 661 | gctagagtat cggagaggaa agcggaattc ctagtgtagc ggtgaaatgc gtagatatta |
| 721 | ggaggaacac cagtggcgaa agcggctttc tggacgacaa ctgacgctga ggcgcgaaag |
| 781 | ccaggggagc aaacgggatt agataccccg gtagtcctgg ccgtaaacga tggatactag |
| 841 | gtgtaggagg tatcgacccc ttctgtgccg gagttaacgc aataagtatc ccgcctgggg |
| 901 | agtacggccg caaggctgaa actcaaagga attgacgggg gcccgcacaa gcggtggagt |
| 961 | atgtggttta attcgacgca acgcgaagaa ccttaccaag ccttgacatt gattgctatg |
| 1021 | gatagagata tccagttcct cttcggagga caagaaaaca ggtggtgcac ggctgtcgtc |
| 1081 | agctcgtgtc gtgagatgtt gggttaagtc ccgcaacgag cgcaacccct atcttctgtt |
| 1141 | accagcggtt cggccgggga ctcaggagag actgccgcag acaatgcgga ggaaggcggg |
| 1201 | gatgacgtca agtcatcatg ccccttatgg cttgggctac acacgtacta caatggctct |
| 1261 | taatagaggg aagcgaagga gcgatccgga gcaaacccca aaaacagagt cccagttcgg |
| 1321 | attgcaggct gcaactcgcc tgcatgaagc aggaatcgct agtaatcgca ggtcagcata |
| 1381 | ctgcggtgaa tacgttcccg ggccttgtac acaccgcccg tcacaccacg aaagtcattc |
| 1441 | acacccgaag ccggtgaggt aaccttttgg agccagccgt ccaaggtggg ggcgatgatt |
| 1501 | ggggtgaagt cgtaacaagg taacc |

### REFERENCES

[1] Spor et al. (2011) Nat Rev Microbiol. 9(4):279-90.
[2] Eckburg et al. (2005) Science. 10; 308(5728):1635-8.
[3] Macpherson et al. (2001) Microbes Infect. 3(12):1021-35
[4] Macpherson et al. (2002) Cell Mol Life Sci. 59(12):2088-96.
[5] Mazmanian et al. (2005) Cell 15; 122(1):107-18.
[6] Frank et al. (2007) PNAS 104(34):13780-5.
[7] Scanlan et al. (2006) J Clin Microbiol. 44(11):3980-8.
[8] Kang et al. (2010) Inflamm Bowel Dis. 16(12):2034-42.
[9] Machiels et al. (2013) Gut. 63(8):1275-83.
[10] WO 2013/050792
[11] WO 03/046580
[12] WO 2013/008039
[13] WO 2014/167338
[14] Goldin and Gorbach (2008) Clin Infect Dis. 46 Suppl 2:S96-100.
[15] Azad et al. (2013) BMJ. 347:f6471.
[16] Deans (2017) Journal of Physiological Anthropology 36:1
[17] Padmanabhan et al. (2013) Standards in Genomic Sciences 8:525-538
[18] Masco et al. (2003) Systematic and Applied Microbiology, 26:557-563.
[19] Srutkova et al. (2011) J. Microbiol. Methods, 87(1):10-6.
[20] Zill et al (2009) Neuroscience 159: 1274-82
[21] Abumaria et al (2008) cell Mol Neurobiol 28: 331-42
[22] Hou et al (2006) Brain Research 1095(1): 154-158
[23] Kim et al. (1987) J Chromatogr. 386:25-35.
[24] Cowen et al (1997) Lancet 349: 915-9.
[25] American Psychiatric Association Diagnostic and statistical manual of mental disorders (5th ed) Washington, D.C.
[26] Luck I (1996) J Clin Psychiatry 57 (Supple. 6): 5-10
[27] Stoddard et al (2011) J Child Adolesc Psychopharmacol. 21: 469-77
[28] Pogarell et al (2003) Biological Psychiatry. 54:1406-1413
[29] Miyamoto-Shinohara et al. (2008) J. Gen. Appl. Microbiol., 54, 9-24.
[30] Cryopreservation and Freeze-Drying Protocols, ed. by Day and McLellan, Humana Press.
[31] Leslie et al. (1995) Appl. Environ. Microbiol. 61, 3592-3597.
[32] Mitropoulou et al. (2013) J Nutr Metab. (2013) 716861.
[33] Kailasapathy et al. (2002) Curr Issues Intest Microbiol. 3(2):39-48.
[34] Handbook of Pharmaceutical Excipients, 2nd Edition, (1994), Edited by A Wade and PJ Weller
[35] Remington's Pharmaceutical Sciences, Mack Publishing Co. (A. R. Gennaro edit. 1985)
[36] US 2016/0067188
[37] Handbook of Microbiological Media, Fourth Edition (2010) Ronald Atlas, CRC Press.
[38] Maintaining Cultures for Biotechnology and Industry (1996) Jennie C. Hunter-Cevera, Academic Press
[39] Strobel (2009) Methods Mol Biol. 581:247-61.
[40] Gennaro (2000) Remington: The Science and Practice of Pharmacy. 20th edition, ISBN: 0683306472.
[41] Molecular Biology Techniques: An Intensive Laboratory Course, (Ream et al., eds., 1998, Academic Press).
[42] Methods In Enzymology (S. Colowick and N. Kaplan, eds., Academic Press, Inc.)
[43] Handbook of Experimental Immunology, Vols. I-IV (D. M. Weir and C. C. Blackwell, eds, 1986, Blackwell Scientific Publications)
[44] Sambrook et al. (2001) Molecular Cloning: A Laboratory Manual, 3rd edition (Cold Spring Harbor Laboratory
Press).
[45] Handbook of Surface and Colloidal Chemistry (Birdi, K. S. ed., CRC Press, 1997)
[46] Ausubel et al. (eds) (2002) Short protocols in molecular biology, 5th edition (Current Protocols).
[47] PCR (Introduction to Biotechniques Series), 2nd ed. (Newton & Graham eds., 1997, Springer Verlag)
[48] Current Protocols in Molecular Biology (F. M. Ausubel et al., eds., 1987) Supplement 30
[49] Smith & Waterman (1981) Adv. Appl. Math. 2: 482-489.
[50] Johnsen et al (2017) Journal of Chromatography A. 1503: 57-64
[51] Michel and Prat (2016) Ann Transl Med. 4(1): 15.

1-19. (canceled)

20. A method for treating or preventing a disorder characterized by serotonin deficiency in a subject in need thereof, comprising administering a composition comprising a bacterial strain of the genus Megasphaera to the subject.

21. The method of claim 20, wherein the disorder is a psychiatric disorder.

22. The method of claim 20, wherein the disorder is selected from the group consisting of depression, anxiety, post-traumatic stress disorder (PTSD) and obsessive compulsive disorder.

23. The method of claim 22, wherein the disorder is depression or PTSD.

24. The method of claim 20, wherein the composition increases the production of serotonin in the subject via a tryptophan hydroxylase pathway.

25. The method of claim 20, wherein the composition increases the expression of tryptophan hydroxylase 1 and/or tryptophan hydroxylase 2 in the subject.

26. The method of claim 20, wherein the composition increases the expression of serotonin transporter in a neuron of the subject.

27. The method of claim 20, wherein the composition increases the secretion of serotonin in the brain of the subject.

28. The method of claim 20, wherein the bacterial strain produces at least one metabolite from the group consisting of butyrate, valeric acid and hexanoic acid, and consumes acetate and/or propionate.

29. The method of claim 28, wherein the bacterial strain produces butyrate, valeric acid and hexanoic acid, and consumes acetate and propionate.

30. The method of claim 20, wherein the bacterial strain has a 16S rRNA sequence that is at least 95% identical to the 16S rRNA sequence of a bacterial strain of the genus Megasphaera.

31. The method of claim 20, wherein the bacterial strain has a 16s rRNA gene sequence that is at least 95% identical to SEQ ID NO:11, 12, 13, 14, 15 or 16.

32. The method of claim 31, wherein the bacterial strain has a 16s rRNA gene sequence of SEQ ID NO:11, 12, 13, 14, 15 or 16.

33. The method of claim 20, wherein the bacterial strain is of Megasphaera massiliensis.

34. The method of claim 20, wherein the bacterial strain has a 16s rRNA gene sequence that is at least 95% identical to SEQ ID NO:1 or 2.

35. The method of claim 20, wherein the bacterial strain has a 16s rRNA gene sequence that is at least 95% identical to SEQ ID NO:2.

36. The method of claim 35, wherein the bacterial strain has a 16s rRNA gene sequence of SEQ ID NO:2.

37. The method of claim 20, wherein the subject has been administered a selective serotonin reuptake inhibitor.

38. The method of claim 20, wherein the composition is administered in combination with a selective serotonin reuptake inhibitor.

39. The method of claim 38, wherein the composition is administered simultaneously with the selective serotonin reuptake inhibitor.

40. The method of claim 38, wherein the selective serotonin reuptake inhibitor is citalopram, dapoxetine, escitalopram, fluoxetine, fluvoxamine, paroxetine, sertraline, or vortioxetine.
